(19) United States
(12) Patent Application Publication
(10) Pub. No.: US 2012/0282615 A1
(43) Pub. Date: Nov. 8, 2012

(54) METHODS OF SCREENING FOR COMPOUNDS FOR USE AS MODULATORS OF LEFT-RIGHT ASYMMETRY IN SCOLIOTIC SUBJECTS AND FOR MONITORING EFFICACY OF AN ORTHOPAEDIC DEVICE

(76) Inventor: Alain MOREAU, Montreal (CA)

(21) Appl. No.: 13/554,217

(22) Filed: Jul. 20, 2012

Related U.S. Application Data
(63) Continuation of application No. 13/153,066, filed on Jun. 3, 2011.
Application No. 13/153,066 is a continuation of application No. 12/553,520, filed on Sep. 3, 2009, now abandoned.
(60) Provisional application No. 61/093,818, filed on Sep. 3, 2008.

(51) Int. Cl. C12Q 1/68

(52) U.S. Cl. 435/6.11; 435/6.12

(57) ABSTRACT

A method of screening for a compound for treating or preventing adolescent idiopathic scoliosis (AIS), said method comprising: (a) contacting a test compound with a paraspinal skin fibroblast or a paraspinal muscle cell sample from the right and/or left side of the spine of a subject; and (b) determining at least one of Nodal, Notch1, Pitx2, Lefty1 and Lefty2's expression and/or activity in the cell sample; wherein the test compound is selected as potentially useful in treating or preventing AIS if at least one of Nodal, Notch1, Pitx2, Lefty1 and Lefty2's expression and/or activity in the cell sample is different in the presence of the test compound as compared to in the absence thereof.

### CROSS REFERENCE TO RELATED APPLICATIONS

This application is a continuation application of U.S. patent application Ser. No. 13/153,066 now pending filed on 3 Jun. 2011, which is itself a continuation application of U.S. patent application Ser. No. 12/553,520 filed 3 Sep. 2009 now abandoned, which claims benefit, under 35 U.S.C. §119(e), of U.S. provisional application Ser. No. 61/093,818 filed Sep. 3, 2008. All documents above are incorporated herein in their entirety by reference.

### STATEMENT REGARDING FEDERALLY SPONSORED RESEARCH OR DEVELOPMENT

N.A.

### FIELD OF THE INVENTION

The present invention relates to methods of screening for compounds for use as modulators of left-right asymmetry in scoliotic subjects.

### SEQUENCE LISTING

This application contains a Sequence Listing in computer readable form entitled 765-sequence_listing-14033.101_ST25, created Jul. 19, 2012 having a size of 141 Ko. The computer readable form is incorporated herein by reference.

### BACKGROUND OF THE INVENTION

Spinal deformities and scoliosis in particular, represent the most prevalent type of orthopaedic deformities in children and adolescents (0.2-6% of the population). Published studies suggest that one to six percent of the population will develop scoliosis. This condition leads to the formation of severe deformities of the spine affecting mainly adolescent girls in number and severity.

At present, the cause of adolescent idiopathic scoliosis (AIS), remains unclear (1,2) and there remains a need to identify children or adolescents at risk of developing AIS or to identify which of the affected individuals are at risk of progression. There also remains a need to identify agents for preventing or treating AIS.

The present description refers to a number of documents, the content of which is herein incorporated by reference in their entirety.

### SUMMARY OF THE INVENTION

The present invention is concerned with the discovery that left-right asymmetry gene expression domains are reversed in adolescent idiopathic scoliosis.

In accordance with an aspect of the present invention, there is provided a method of screening for a compound for treating or preventing adolescent idiopathic scoliosis (AIS), said method comprising: (a) contacting a test compound with a paraspinal skin fibroblast or a paraspinal muscle cell sample from the right and/or left side of the spine of a subject; and (b) determining at least one of Nodal, Notch1, Pitx2, Lefty1 and Lefty2's expression and/or activity in the cell sample; wherein the test compound is selected as potentially useful in treating or preventing AIS if at least one of Nodal, Notch1, Pitx2, Lefty1 and Lefty2's expression and/or activity in the cell sample is different in the presence of the test compound as compared to in the absence thereof.

As used herein the term “different” in the context of the expression of a gene refers to a higher or a lower expression of the gene.

As used herein a “higher” or “increased” expression level refers to a level of expression or activity in a sample (e.g., AIS test sample) which is at least 15% higher, in an embodiment at least 25% higher, in a further embodiment at least 40% higher; in a further embodiment at least 50% higher, in a further embodiment at least 100% higher (i.e. 2-fold), in a further embodiment at least 200% higher (i.e. 3-fold), in a further embodiment at least 300% higher (i.e. 4-fold), in a further embodiment at least 400% higher (i.e. 5-fold), in a further embodiment at least 500% higher (i.e. 6-fold), in a further embodiment at least 900% higher (i.e. 10-fold), etc. relative to the reference level (e.g., in a corresponding control sample).

As used herein a “lower” or “decreased” expression level refers to a level of expression or activity in a sample (e.g., AIS test sample) which is at least 15% lower, in an embodiment at least 25% lower, in a further embodiment at least 40% lower; in a further embodiment at least 50% lower, in a further embodiment at least 100% lower (i.e. 2-fold), in a further embodiment at least 200% lower (i.e. 3-fold), in a further embodiment at least 300% lower (i.e. 4-fold), in a further embodiment at least 400% lower (i.e. 5-fold), in a further embodiment at least 500% lower (i.e. 6-fold), in a further embodiment at least 900% lower (i.e. 10-fold), etc. relative to the reference level (e.g., in a corresponding control sample).

The terms “treat/treating/treatment” and “prevent/preventing/prevention” as used herein, refer to eliciting the desired biological response, i.e., a therapeutic and prophylactic effect, respectively. In accordance with the subject invention, the therapeutic effect can be a decrease/reduction of the cobb's angle of the subject, following administration of the agent/composition of the invention. In accordance with the invention, a prophylactic effect may comprise a complete or partial avoidance/inhibition or a delay of scoliosis, following administration of the agent that modulates Nodal, Notch1, Pitx2, Lefty1 and Lefty2's expression and/or activity (or of a composition comprising the agent).

In a specific embodiment of the method, the test compound is selected as potentially useful in treating AIS in a subject having a right thoracic curve when i) at least one of Nodal, Notch1, Pitx2, Lefty1 and Lefty2's expression in the cell sample from the left side of the spine is higher in the presence of the test compound as compared to in the absence thereof; or when ii) at least one of Nodal, Notch1, Pitx2, Lefty1 and Lefty2's expression in the cell sample from the right side of the spine is lower in the presence of the test compound as compared to in the absence thereof; or when iii) both i) and ii).

In another specific embodiment of the method, the test compound is selected as potentially useful in treating AIS in a subject having a left thoracic curve when at least one of Nodal, Notch1, Pitx2, Lefty1 and Lefty2's expression in the cell sample from the left side of the spine is lower in the presence of the test compound as compared to in the absence thereof.

In another specific embodiment of the method, the cell sample is from a subject having AIS. In another specific embodiment of the method, the cell sample is from a subject that is a likely candidate for developing AIS. In another specific embodiment of the method, the cell sample is from a subject exhibiting a right thoracic curve. In another specific embodiment of the method, the cell sample is from a subject exhibiting a left thoracic curve. In another specific embodiment of the method, the cell sample is from a bipedal C57BI/6j mouse.

In accordance with an aspect of the present invention, there is provided a method of screening for a compound for treating or preventing adolescent idiopathic scoliosis (AIS), said method comprising: (a) administering a test compound to a scoliotic bipedal C57BI/6j mouse; and (b) determining at least one of Nodal, Notch1, Pitx2, Lefty1 and Lefty2's expression and/or activity in a paraspinal skin fibroblast or a paraspinal muscle cell sample from the mouse; wherein the test compound is selected as potentially useful in treating or preventing AIS if at least one of Nodal, Notch1, Pitx2, Lefty1 and Lefty2's expression and/or activity in the cell sample is different in the presence of the test compound as compared to in the absence thereof. In accordance with an aspect of the present invention, the compound selected in the in vitro method may further be validated in the scoliotic bipedal C57BI/6j mouse to determine prevention or treatment of adolescent idiopathic scoliosis (AIS) by determining whether scoliosis is prevented or treated.

In accordance with another aspect of the present invention, there is provided a method of monitoring efficacy of an orthopaedic device in a subject having adolescent idiopathic scoliosis (AIS) comprising a) measuring expression of at least one of Nodal, Notch1, Pitx2, Lefty1 and Lefty2 in a sample of paraspinal muscle cells from at least one side of the apex of the main scoliosis curve of the subject before having installed the device on the subject; and b) repeating the measure of step a) after having installed the device on the subject, wherein a difference of expression of at least one of Nodal, Notch1, Pitx2, Lefty1 and Lefty2 between steps a) and b) provides an indication on the efficacy of the device.

In a specific embodiment of the method, the main scoliosis curve is a right thoracic curve, and a lower expression of at least one of Nodal, Notch1, Pitx2, Lefty1 and Lefty2 in the sample from the right side of the apex in step b) as compared to that in step a) is an indication that the device is efficient. In another specific embodiment of the method, the main scoliosis curve is a right thoracic curve, and a higher expression of at least one of Nodal, Notch1, Pitx2, Lefty1 and Lefty2 in the sample from the left side of the apex in step b) as compared to that in step a) is an indication that the device is efficient.

In accordance with still another aspect of the present invention, there is provided a method of identifying a mutation contributing to adolescent idiopathic scoliosis (AIS), comprising comparing the nucleotide sequence of a gene of at least one of Nodal, Notch1, lefty1, Lefty2 and Pitx2 from a subject having AIS with that of the corresponding gene in a control subject, wherein the presence of a mutation in the gene of the subject is an indication that the mutation contributes to AIS.

In a specific embodiment of the method, the nucleotide sequence is that of Nodal. In another specific embodiment of the method, the nucleotide sequence is that of Notch1. In another specific embodiment of the method, the nucleotide sequence is that of Lefty1. In another specific embodiment of the method, the nucleotide sequence is that of Lefty2. In another specific embodiment of the method, the nucleotide sequence is that of Pitx2.

In accordance with still another aspect of the present invention, there is provided a method for identifying at least one mutation directly or indirectly contributing to adolescent idiopathic scoliosis (AIS), comprising analyzing the sequence of a gene whose product directly or indirectly modulates Nodal, Notch1, Pitx2, Lefty1 and/or Lefty2's expression, wherein the gene is from a subject having AIS, or analyzing the sequence of the gene's product, wherein the presence of a mutation in the gene or in the gene's product is an indication that the mutation contributes to AIS in the subject.

In a specific embodiment, the method comprises detecting in the subject a mutation in a gene who's product directly or indirectly modulates Nodal, Notch1, Pitx2, Lefty1 and/or Lefty2's asymmetrical expression in paraspinal muscle cells.

In accordance with a further aspect of the present invention, there is provided a method for determining whether a test compound is useful for the prevention and/or treatment of adolescent idiopathic scoliosis, said method comprising: (a) contacting said test compound with a cell comprising a first nucleic acid comprising a transcriptionally regulatory element normally associated with a Nodal, Notch1, Pitx2, Lefty1 or Lefty2 gene, operably linked to a second nucleic acid comprising a reporter gene encoding a reporter protein; and (b) determining whether the reporter gene expression and/or reporter protein activity is modified in the presence of said test compound; wherein said difference in reporter gene expression and/or reporter protein activity is indicative that said test compound may be used for prevention and/or treatment of adolescent idiopathic scoliosis.

The articles “a,” “an” and “the” are used herein to refer to one or to more than one (i.e., to at least one) of the grammatical object of the article.

The term “including” and “comprising” are used herein to mean, and re used interchangeably with, the phrases “including but not limited to” and “comprising but not limited to”.

The terms “such as” are used herein to mean, and is used interchangeably with, the phrase “such as but not limited to”.

As used herein the terms “likely candidate for developing adolescent idiopathic scoliosis” include children of which a least one parent has adolescent idiopathic scoliosis. Among other factors, age (adolescence), gender and heredity (i.e. born from a mother or father having a scoliosis) are factors that are known to contribute to the risk of developing a scoliosis and are used to a certain degree to assess the risk of developing AIS. In certain subjects, scoliosis develops rapidly over a short period of time to the point of requiring a corrective surgery (often when the deformity reaches a Cobb's angle ≧50°). Current courses of action available from the moment AIS is diagnosed (when scoliosis is apparent) include observation (when Cobb's angle is around 10-25°), orthopaedic devices (when Cobb's angle is around 25-30°), and surgery (over 45°). A more reliable determination of the risk of progression could enable to 1) select an appropriate diet to remove certain food products identified as contributors to scoliosis; 2) select the best therapeutic agent; 3) select the least invasive available treatment such as postural exercises, orthopaedic device, or less invasive surgeries or surgeries without fusions (a surgery that does not fuse vertebra and preserves column mobility). The present invention encompasses selecting the most efficient and least invasive known preventive actions or treatments in view of the determined risk of developing AIS.

As used herein, the terms “severe AIS” refers to a scoliosis characterized by Cobb's angle of 45° or more.

As used herein, the term “Pitx2 expression” is used to refer Pitx2 transcription and/or translation. In a specific embodiment, it refers to Pitx2 transcription.

As used herein, the terms “Lefty1 gene” or “Lefty2 gene” or “Nodal gene” or “Notch1 gene” or “Pitx2 gene” refers to genomic DNA encoding sequences comprising those sequences referred to in GenBank by GeneID numbers referred to in Figures presented herein for instance. The description of the various aspects and embodiments of the invention is provided with reference to exemplary nucleic acids and polypeptides. Such reference is meant to be exemplary only and the various aspects and embodiments of the invention are also directed to other genes that express alternate Lefty1, Lefty2, Nodal, Notch1 or Pitx2 nucleic acids, such as mutant Lefty1, Lefty2, Nodal, Notch1 or Pitx2 nucleic acids, splice variants of Lefty1, Lefty2, Nodal, Notch1 or Pitx2 nucleic acids, Lefty1, Lefty2, Nodal, Notch1 or Pitx2 variants from species to species or subject to subject.

As used herein, the term “Lefty1 expression” is used to refer to Lefty1 transcription and/or translation. In a specific embodiment, it refers to Lefty1 transcription.

As used herein, the term “Lefty2 expression” is used to refer to Lefty2 transcription and/or translation. In a specific embodiment, it refers to Lefty2 transcription.

As used herein, the term “Nodal expression” is used to refer to Nodal transcription and/or translation. In a specific embodiment, it refers to Nodal transcription.

As used herein, the term “Notch1 expression” is used to refer to Notch1 transcription and/or translation. In a specific embodiment, it refers to Notch1 transcription.

As used herein the terms “Lefty1 activity”, “Lefty2 activity” , “Nodal activity”, “Notch1 activity” and “Pitx2 activity” refer to detectable enzymatic, biochemical or cellular activity attributable to Lefty1, Lefty2, Nodal, Notch1, and Pitx2 gene product respectively. Without being so limited, Nodal, Lefty1, Lefty2 et Pitx2 act as transcription factors and regulatory molecule (Nodal). During development, it is known that Nodal positively activates the expression of Pitx2 to define the left domain in normal embryo. Lefty2 is activated by Nodal while in return accumulation of Lefty2 will lead to a repression of Nodal (retroactive feedback loop). Notch1 is a membranous receptor and upon activation through the binding of specific ligands (e.g., Delta, Serrate and Jagged, there are for some of these ligands more than one family members) will be cleaved and translocated in the nucleus to activate specific genes.

As used herein, the terms “the main scoliosis curve” is meant to refer to the spinal deformity having the more severe angulation (Cobb angle) when more than one curve are detected in a subject.

As used herein, the terms “apex of the main scoliosis curve” is meant to refer to the maximal convexity/concavity or the tip of the curve.

As used herein, the terms “orthopaedic device” is meant to refer to any instrument meant to correct or prevent the scoliosis. Without being so limited it includes orthopaedic braces including those commercialized under the trademarks Boston™, Cheneau™, SpineCor™, Providence™, etc.; surgical devices such as metal rods, screws, hooks, strings; devices meant to maximize spine mobility such as Orthobiom™, and various staples; and apparatuses of the distracter type fixable on ribs or pelvis.

As used herein the terms “risk of developing AIS” and “risk of progression of AIS” are used interchangeably and refer to a genetic or metabolic predisposition of a subject to develop a scoliosis (i.e. spinal deformity) and/or a more severe scoliosis at a future time.

As used herein the term “subject” is meant to refer to any mammal including human, mice, rat, dog, cat, pig, monkey, horse, etc. In a particular embodiment, it refers to a human. In another particular embodiment, it refers to a horse and more specifically a racing horse.

As used herein the terms “control sample” are meant to refer to a sample from a subject without AIS or familial history of AIS (control subject). In a particular embodiment, the control sample is from a subject without scoliosis and familial history of scoliosis. In another particular embodiment, the control subject has congenital scoliosis involving a structural defect. In addition to paraspinal muscle cells, it is expected that paraspinal skin fibroblasts could also be used as sample from the subject and control (6).

Without being so limited, cells where Pitx2 is known to be expressed include osteoblasts, skeletal muscle cells, extraocular muscle cells, chondrocytes, periumbelical skin cells and fibroblasts.

Without being so limited, cells where Lefty1 is known to be expressed include cells from the hypothalamus, extraocular muscle, brain, heart, kidney, liver, lung, muscle, neuromuscular junction, spleen and bone.

Without being so limited, cells where Lefty2 is known to be expressed include cells from the hypothalamus, extraocular muscle, brain, heart, kidney, liver, lung, muscle and spleen.

Without being so limited, cells where Nodal is known to be expressed include cells from the hypothalamus, extraocular muscle, brain, heart, kidney, liver, lung, muscle and spleen.

Without being so limited, cells where Notch1 is known to be expressed include cells from the hypothalamus, extraocular muscle, brain, heart, kidney, liver, lung, muscle, spleen.

The present invention encompasses methods for identifying a mutation in a gene. Such methods include, without being so limited, Wave nucleic acid fragment analysis (dHPLC) and direct sequencing on PCR fragments amplified from genomic DNA isolated from subjects.

The present invention also relates to methods for the determination of the level of expression of transcripts or translation products of a single gene such as nodal, Notch1, Pitx2, Lefty-1 and/or Lefty2. The present invention therefore encompasses any known method for such determination including real time PCR and competitive PCR, Northern blots, nuclease protection, plaque hybridization and slot blots.

The present invention also concerns isolated nucleic acid molecules including probes. In specific embodiments, the isolated nucleic acid molecules have no more than 300, or no more than 200, or no more than 100, or no more than 90, or no more than 80, or no more than 70, or no more than 60, or no more than 50, or no more than 40 or no more than 30 nucleotides. In specific embodiments, the isolated nucleic acid molecules have at least 20, or at least 30, or at least 40 nucleotides. In other specific embodiments, the isolated nucleic acid molecules have at least 20 and no more than 300 nucleotides. In other specific embodiments, the isolated nucleic acid molecules have at least 20 and no more than 200 nucleotides. In other specific embodiments, the isolated nucleic acid molecules have at least 20 and no more than 100 nucleotides. In other specific embodiments, the isolated nucleic acid molecules have at least 20 and no more than 90 nucleotides. In other specific embodiments, the isolated nucleic acid molecules have at least 20 and no more than 80 nucleotides. In other specific embodiments, the isolated nucleic acid molecules have at least 20 and no more than 70 nucleotides. In other specific embodiments, the isolated nucleic acid molecules have at least 20 and no more than 60 nucleotides. In other specific embodiments, the isolated nucleic acid molecules have at least 20 and no more than 50 nucleotides. In other specific embodiments, the isolated nucleic acid molecules have at least 20 and no more than 40 nucleotides. In other specific embodiments, the isolated nucleic acid molecules have at least 20 and no more than 30 nucleotides. In other specific embodiments, the isolated nucleic acid molecules have at least 30 and no more than 300 nucleotides. In other specific embodiments, the isolated nucleic acid molecules have at least 30 and no more than 200 nucleotides. In other specific embodiments, the isolated nucleic acid molecules have at least 30 and no more than 100 nucleotides. In other specific embodiments, the isolated nucleic acid molecules have at least 30 and no more than 90 nucleotides. In other specific embodiments, the isolated nucleic acid molecules have at least 30 and no more than 80 nucleotides. In other specific embodiments, the isolated nucleic acid molecules have at least 30 and no more than 70 nucleotides. In other specific embodiments, the isolated nucleic acid molecules have at least 30 and no more than 60 nucleotides. In other specific embodiments, the isolated nucleic acid molecules have at least 30 and no more than 50 nucleotides. In other specific embodiments, the isolated nucleic acid molecules have at least 30 and no more than 40 nucleotides.

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally known (62,63). Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

As used herein the terms “detectably labeled” refer to a marking of a probe in accordance with the presence invention that will allow the detection of the mutation of the present invention. Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods. Non-limiting examples of labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma 32P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

The present invention also relates to methods of selecting compounds. As used herein the term “compound” is meant to encompass natural, synthetic or semi-synthetic compounds, including without being so limited chemicals, macromolecules, cell or tissue extracts (from plants or animals), nucleic acid molecules, peptides, antibodies and proteins.

The present invention also relates to arrays. As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

As used herein “array of nucleic acid molecules” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

As used herein “solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Any known nucleic acid arrays can be used in accordance with the present invention. For instance, such arrays include those based on short or longer oligonucleotide probes as well as cDNAs or polymerase chain reaction (PCR) products (52). Other methods include serial analysis of gene expression (SAGE), differential display, (53) as well as subtractive hybridization methods (54), differential screening (DS), RNA arbitrarily primer (RAP)-PCR, restriction endonucleolytic analysis of differentially expressed sequences (READS), amplified restriction fragment-length polymorphisms (AFLP).

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see 64 for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1 ×SSC at 55 to 60° C.

Washing with a solution containing tetramethylammonium chloride (TeMAC) could allow the detection of a single mismatch using oligonucleotide hybridyzation since such mismatch could generate a 10° C. difference in the annealing temperature. The formulation to determine the washing temperature is Tm (° C.)=]−682 (L⁻¹)+97 where L represents the length of the oligonucleotide that will be used for the hybridization. When the oligonucleotide of the present invention has a length of 20 nucleotides: the hybridization is performed 5° C. below the Tm which is calculated using the formula above at 62.9° C. In principle, a single mismatch will generate a 10° C. drop in the annealing so that a temperature of 57° C. should only detect mutants harbouring the T mutation. Such conditions are high stringency conditions appropriate to identify a single nucleotide mutation in the 20 nucleotides probes of the present invention (56).

The present invention relates to a kit for screening for direct or indirect modulators of Pitx2, Nodal, Notch1, Lefty-1 and/or Lefty2 comprising an isolated nucleic acid, a protein or a ligand such as an antibody in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the subject sample (DNA genomic nucleic acid, cell sample or blood samples), a container which contains in some kits of the present invention, the probes used in the methods of the present invention, containers which contain enzymes, containers which contain wash reagents, and containers which contain the reagents used to detect the extension products. The present invention also relates to a kit comprising the antibodies which are specific to pitx1 repressors. Kits of the present invention may also contain instructions to use these probes and or antibodies to identify mutations in Pitx2, Nodal, Notch1, Lefty-1 and/or Lefty2 or direct or indirect modulators of these genes.

### Antibodies

Both monoclonal and polyclonal antibodies directed to Pitx2, Nodal, Notch1, Lefty-1 or Lefty2 are included within the scope of this invention as they can be produced by well established procedures known to those of skill in the art. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents in A) radiography of scoliotic deformities of 8 bipedal C57BI/6J mice. Non-invasive analysis of scoliosis progression was performed using a Faxitron™ X-rays cabinet under anaesthesia. Note that bipedal C57BI/6j females (upper panels #704, 706, 708) developed more profound scoliosis when compared to scoliotic males (lower panels #903, 907). Note also that female #707 and males #906 and #919 are non scoliotic; and B) a photography of a bipedal C57 BI/6j mouse;

FIG. 2 presents the left-right expression analysis by RT-PCR of the genes Nodal and Lefty2 in paraspinal muscles cells at the apex of the main curve of bipedal C57BI/6J scoliotic mouse #706 (exhibiting a right thoracic curve), scoliotic mouse #708 (exhibiting a left thoracic curve) and non scoliotic mouse #707. (3-actin expression levels were used as internal controls;

FIG. 3 presents in A) an x-ray image of the scoliosis in a human having AIS with a right thoracic curve; and in B) a schematic representation of a right thoracic curve in a scoliotic patient and left and right regions of this curve: above the main curve (H), below the main curve (L) and at the apex of the main curve (A);;

FIG. 4 presents in A) the left-right expression of Lefty 2, Pitx2 and β-actin as a control from intraoperatively taken biopsies of paraspinal muscles above (H), below (L) and at the apex (A) of the main curve on each side of the spine of a human scoliotic subject displaying a right thoracic curve; and in B) these expressions normalized over the β-actin control for Lefty-2 and for Pitx2;

FIG. 5 presents the mean relative quantification of Lefty1 expression on the left side of the curve compared to that on the right side in AIS subjects. Paraspinal muscle biopsies were taken at the apex of the left and right sides of the vertebral column of AIS patients (n=6) and control subject (n=1). RNAs prepared from the biopsies were used to perform real-time quantitative PCR in order to assess changes in expression of LR-asymmetry genes. These graphs show mean relative quantification of Lefty1 expression on the left side (AL) compared to the right side (AR) in AIS patients (panel A) and control subjects (panel B). As shown in control subject (panel B), Lefty1 is normally more highly expressed on the left side of the apex, while in AIS patients, Lefty1 is more highly expressed on the right side. This difference is statistically significant (Panel A: p=0.030);

FIG. 6 presents the mean relative quantification of Lefty2 expression on the left side of the curve compared to that on the right side in AIS subjects. Paraspinal muscle biopsies were taken at the apex of the left and right sides of the vertebral column of AIS patients (n=6) and control subject (n=1). RNAs prepared from the biopsies were used to perform real-time quantitative PCR in order to assess changes in expression of LR-asymmetry genes. These graphs show mean relative quantification of Lefty2 expression on the left side (AL) compared to the right side (AR). As shown in control subject (panel B), Lefty2 is normally more highly expressed on the left side of the apex, while in AIS patients (panel A), Lefty2 is more highly expressed on the right side. This difference is statistically significant (p=0.0019);

FIG. 7 presents the mean relative quantification of Pitx2 expression on the left side of the curve compared to that on the right side in AIS subjects. Paraspinal muscle biopsies were taken at the apex of the left and right sides of the vertebral column of AIS patients (n=6) and control subject (n=1). RNAs prepared from the biopsies were used to perform real-time quantitative PCR in order to assess changes in expression of LR-asymmetry genes. These graphs show mean relative quantification of Pitx2 expression on the left side (AL) compared to the right side (AR). As shown in the control subject (panel B), Pitx2 is normally more highly expressed on the left side of the apex, while in AIS patients (panel A), it is more highly expressed on the right side;

FIG. 8 presents the mean relative quantification of Notch1 expression on the left side of the curve compared to that on the right side in AIS subjects. Paraspinal muscle biopsies were taken at the apex of the left and right sides of the vertebral column of AIS patients (n=6) and control subject (n=1). RNAs prepared from the biopsies were used to perform real-time quantitative PCR in order to assess changes in expression of LR-asymmetry genes. These graphs show mean relative quantification of Notch1 expression, a known regulator of Nodal, on the left side (AL) compared to that on the right side (AR). As shown in the control subject (panel B), Notch1 is normally more highly expressed on the left side of the apex, while in AIS patients (panel A), it is more highly expressed on the right side;

FIG. 9 presents the mean relative quantification of Nodal expression on the left side of the curve compared to that on the right side in AIS subjects. Paraspinal muscle biopsies were taken at the apex of the left and right sides of the vertebral column of AIS patients (n=3). RNAs prepared from the biopsies were used to perform real-time quantitative PCR in order to assess changes in expression of LR-asymmetry genes. This graph shows mean relative quantification of Nodal expression, on the left side (AL) compared to that on the right side (AR). As shown in AIS patients Nodal is more highly expressed on the right side;

FIG. 10 shows the sequence of the human Pitx2 mRNA, transcript variant 1 (SEQ ID NO: 1);

FIG. 11 shows the sequence of the human Pitx2 mRNA, transcript variant 2 (SEQ ID NO: 2);

FIG. 12 shows the sequence of the human Pitx2 mRNA, transcript variant 3 (SEQ ID NO: 3);

FIG. 13 shows the sequence of the human Pitx2 amino acid sequences for (A) isoform a (SEQ ID NO: 4), (B) isoform b (SEQ ID NO: 5), and (C) isoform c (SEQ ID NO: 6);

FIG. 14 shows the sequence of the mouse Pitx2 mRNA, transcript variant 1 (SEQ ID NO: 7);

FIG. 15 shows the sequence of the mouse Pitx2 mRNA, transcript variant 2 (SEQ ID NO: 8);

FIG. 16 shows the sequence of the mouse Pitx2 mRNA, transcript variant 3 (SEQ ID NO: 9);

FIG. 17 shows mouse Pitx2 amino acid sequence for (A) isoform a (SEQ ID NO: 10), (B) isoform b (SEQ ID NO: 11), and (C) isoform c (SEQ ID NO: 12);

FIG. 18 shows the sequence of the human Lefty1 mRNA (SEQ ID NO: 13);

FIG. 19 shows the human Lefty1 amino acid sequence (SEQ ID NO: 14);

FIG. 20 shows the sequence of the mouse Lefty1 mRNA (SEQ ID NO: 15);

FIG. 21 shows the mouse Lefty1 amino acid sequence (SEQ ID NO: 16);

FIG. 22 shows the sequence of the human Lefty2 mRNA (SEQ ID NO: 17);

FIG. 23 shows the human Lefty2 amino acid sequence (SEQ ID NO: 18);

FIG. 24 shows the sequence of the mouse Lefty2 mRNA (SEQ ID NO: 19);

FIG. 25 shows the mouse Lefty2 amino acid sequence (SEQ ID NO: 20);

FIG. 26 shows the human Notch1 mRNA sequence (SEQ ID NO: 21);

FIG. 27 shows the human Notch1 amino acid sequence (SEQ ID NO: 22);

FIG. 28 shows the mouse Notch1 mRNA sequence (SEQ ID NO: 23);

FIG. 29 shows the mouse Notch1 amino acid sequence (SEQ ID NO: 24);

FIG. 30 shows the human Nodal mRNA sequence (SEQ ID NO: 25);

FIG. 31 shows the human Nodal amino acid sequence (SEQ ID NO: 26); and

FIG. 32 shows A) the mouse Nodal nucleotide sequence (SEQ ID NO: 27) and B) amino acid sequence (SEQ ID NO:28).

### DESCRIPTION OF ILLUSTRATIVE EMBODIMENTS

A study of molecular changes occurring in paraspinal muscles of AIS subjects showed a reversal in Nodal, Lefty-1, Lefty2 and Pitx2 expression. These left-side restricted genes were expressed on the right side of AIS subjects exhibiting a right thoracic curve, while those genes were expressed on the left side of control subjects. These changes occur at the apex of the curve.

Similar results were obtained in a study performed in scoliotic bipedal C57BI6/j mice. They also showed a reversal of left-right asymmetrical expression in Nodal, Lefty2 and Pitx2 expression domains. Furthermore, in the rare event of left thoracic scoliosis, these genes were still expressed on the left side although they were over expressed.

It was thus discovered that Nodal, Notch1, Lefty1, Lefty2 and Pitx2 normally expressed on the left side are expressed on the right side of the paraspinal musculature of AIS subjects.

This observation could explain the high prevalence of right thoracic curves in AIS patients and in the scoliotic C57BI/6j mice model. Conversely, this may also explain why left thoracic curves are rarer because such L-R reversal is incomplete or absent.

The present invention is illustrated in further details by the following non-limiting examples.

### Mice

C57BI/6j mice were served as wild-type control mice (Charles-River, Wilmington, Mass., USA). The C57BI6/6j mouse strain was used because it is naturally deficient in melatonin (8), and exhibits high circulating OPN levels (9);. The mice of this strain develop scoliosis when they are maintained in a bipedal state (10). Bipedal surgeries were performed after weaning by amputation of the forelimbs and tail under anesthesia as reported previously (10).

### Human Subjects

Informed consent was obtained from all study participants as approved by each individual and collective Institutional Review Board (Ste-Justine University Hospital, Montreal's Children Hospital and The Shriners Hospital for Children all located in Montreal). All individuals were screened through a series of steps including history and clinical data, assuring the idiopathic nature of the problem. This was followed by a review of spinal radiographs. A person was deemed to be affected by AIS if history and physical examination were consistent with the diagnosis of idiopathic scoliosis and a minimum of a ten degree curvature in the coronal plane with vertebral rotation was found on the radiograph. Other patients (congenital scoliosis caused by a structural defect such as hemivertebras or missing vertebras) were used as controls.

TABLE 1
clinical characteristic of AIS patients.

| | | | | | Cobb′s |
|---|---|---|---|---|---|
| # | Gender | Age | Diagnosis | Curve | angle |
| 1475 | M | 14 | Kyphosis | Kyphosis | 72 |
| 1444 | F | 15 | AIS | RTLL | 52-51 |
| 1473 | F | 15 | AIS | RT | 35 |
| 1477 | F | 15 | AIS | RTLL | 62-65 |
| 1493 | F | 16 | AIS | RTLL | 55-46 |
| 1510 | F | 14 | AIS | RT | 42 |
| 1514 | F | 14 | AIS | RTLL | 72-44 |

RTLL = double major curve, right thoracic, left lumbar
RT = right thoracic

Biopsies from Mice, AIS Patients and Controls

Paraspinal muscle biopsies were taken intraoperatively on each side of the spine of the vertebral column of mice (50 bipedal C57BI/6 mice (20 scoliotic mice)) and of AIS patients (Table 1 above). At the time of the surgery, the surgeon was requested to perform small biopsies at the apex of the curve and above and below (about 1 or 2 vertebras). A paraspinal fragment of 1 cm³ or less was taken and kept in culture media upon its arrival in the lab. On reception, the samples were immediately frozen in liquid nitrogen and conserved at −80° C. until processed for RNA extraction.

### X-Ray Radiography

All mice underwent complete radiographic examination under anesthesia using a Faxitron™ X-rays apparatus (Faxitron X-rays Corp. Wheeling, Ill., USA) every two weeks starting at the age of six weeks. Anteroposterior X-rays were taken and each digital image was evaluated subsequently for the presence of scoliosis. Cobb's angle threshold value of 10° or higher was retained as a significant scoliotic condition. For humans, a person was deemed to be affected if history and physical examination were consistent with the diagnosis of idiopathic scoliosis and a minimum of a ten degree curvature in the coronal plane with vertebral rotation was found by radiograph.

### Total RNA Isolation an RT-PCR

RNA was extracted using Trizol™ reagent (Invitrogen, Burlington, ON). Total RNA were reverse transcribed in a final volume of 20 μL using Thermoscript™ reverse transcription kit (Invitrogen) as described by the manufacturer. Reverse transcribed samples were stored at −20° C. until assayed.

The RNA obtained from the biopsies was used for cDNA synthesis performed with the Invitrogen Thermoscript™ RT-PCR system and the respective protocol in the following conditions: Enzyme used for β-actin amplification: Taq DNA polymerase from Invitrogen™. PCR conditions: 95° C. 5 minutes Hot start (1 cycle). Following three reactions (32 cycles): 94° C. 45 Seconds Denaturation; 55° C. 45 Seconds Primer annealing; 72° C. 1 minute Elongation; 72° C. 2 minutes Last elongation (1 cycle); 4° C. 20 minutes pause; Duration: 2 hours 42 minutes. The quality of the cDNA was tested by amplifying 233 bp fragment of human beta-actin using the sense primer 5′-GGAAATCGTGCGTGACAT-3′(SEQ ID NO: 29) and antisense primer 5′-TCATGATGGAGTTGAATGTAGTT-3′ (SEQ ID NO: 30). For quantitative analysis, all amplifications were normalized against that of the housekeeping gene β-actin. PCR amplified product were separated on 1.5% agarose gel and visualized by ethidium bromide staining.

### Expression Analysis of Mouse Pitx2:

Coding region of mouse Pitx2 588 pb in length was amplified from the cDNA using the sense primer 5′-CGCGGGGATCCGAGGACTG-3′ (SEQ ID NO: 31) and the antisense primer 5′-TACACAGGATGGGTCGTACA-3′ (SEQ ID NO: 32) under the following PCR conditions: Enzyme used: Pfx DNA polymerase from Invitrogen™. PCR conditions: 95° C. 10 minutes hot start (1 cycle); Following three reactions (34 cycles): 94° C. 45 Seconds Denaturation; 69° C. 45 Seconds Primer annealing; 72° C. 1 minute Elongation; 72° C. 2 minutes Last elongation (1 cycle); 4° C. 20 minutes; 4° C. Pause; Duration: 2 hours 34 minutes 11 seconds.

### Expression Analysis of Mouse Lefty2:

Coding region of mouse Lefty2 593 bp in length was amplified from the cDNA using the sense primer 5′-CGTGAGGTCCCAGTATGTGG-3′ (SEQ ID NO: 33) and the antisense primer 5′-GTAGTCCTTGAGGTCCAGCG-3′ (SEQ ID NO: 34) under the following PCR conditions: Enzyme used: HiFi Taq DNA polymerase from Invitrogen™. PCR conditions: 95° C. 5 minutes hot start (1 cycle); Following three reactions (35 cycles): 94° C. 45 seconds for Denaturation; 60° C. 45 seconds for primer annealing; 68° C. 1.2 minute (80 seconds) for Elongation; 72° C. Finally, 2 minutes for a last elongation at 68° C. (1 cycle); 4° C. 20 minutes.

### Expression Analysis of Mouse Nodal:

Coding region of mouse Nodal 544bp in length was amplified from the cDNA using the sense primer 5′-GTGACCGGACAGAACTGGAC-3′ (SEQ ID NO: 35) and the antisense primer 5′-CTGTCTGGCAAATGATGTCG-3′ (SEQ ID NO: 36) under the following PCR conditions: Enzyme used: Pfx DNA polymerase from Invitrogen™. PCR conditions: 95° C. 5 minutes hot start (1 cycle); Following three reactions (34 cycles): 94° C. 45 seconds for denaturation; 65° C. 45 seconds for primer annealing; 68° C. 1.2 minute (80 seconds) for Elongation; Finally 68° C. 2 minutes for a last elongation (1 cycle); 4° C. 20 minutes.

### ABI Gene Expression Assay

Gene expression level was determined using primer and probe sets from Applied Biosystems (ABI Gene Expression Assays, http://www.appliedbiosystems.com/). PCR reactions for 384 well plate formats were performed using 2 μl of cDNA samples (20-50 ng), 5 μl of the TaqMan™ Universal PCR Master Mix (Applied Biosystems, Calif.), 0.5 μl of the TaqMan™ Gene Expression Assays (20×) and 2.5 μl of water in a total volume of 10 μl. The following pre-developed TaqMan™ assays were used as endogenous control: GAPDH (glyceraldehyde-3-phosphate dehydrogenase), HPRT (hypoxanthine guanine phosphoribosyl transferase), ACTB (Beta actin) and 18S (ribosomal RNA).

### Detection and Analysis

The ABI PRISM™ 7900HT Sequence Detection System (Applied Biosystems) was used to detect the amplification level and was programmed to an initial step of 10 minutes at 95° C., followed by 45 cycles of 15 seconds at 95° C. and 1 minute at 60° C. All reactions were run in triplicate and the average values were used for quantification. The human GAPDH (glyceraldehyde-3-phosphate dehydrogenase), ACTB (Beta Actin) or 18S ribosomal RNA were used as endogenous controls.

The relative quantification of target genes was determined by using the ΔΔCT method. Briefly, the Ct (threshold cycle) values of target genes were normalized to an endogenous control gene (GAPDH) (ΔCT=Ct_{target}−Ct_{GAPDH}) and compared with a calibrator: ΔΔCT=ΔCt_{Sample}−ΔCt_{Calibrator}. Relative expression (RQ) was calculated using the Sequence Detection System (SDS) 2.2.2 software (Applied Biosystems) and the formula RQ=2^{-ΔCT}.

TABLE 2
Primers for real time PCR

| GENES | Forward | Reverse |
|---|---|---|
| Human | aggttcagccagagcttcc | caccagcaggtgtgtgct |
| LEFTY | (SEQ ID NO: 37) | (SEQ ID NO: 38) |
| 1 | | |
| Human | cctggacctcagggactatg | atcccctgcaggtcaatgta |
| LEFTY | (SEQ ID NO: 39) | (SEQ ID NO: 40) |
| 2 | | |
| Human | agacatcatccgcagccta | caaaagcaaacgtccagttct |
| NODAL | (SEQ ID NO: 41) | (SEQ ID NO: 42) |
| Human | ccttacggaagcccgagt | ccgaagccattcttgcata |
| PITX2 | (SEQ ID NO: 43) | (SEQ ID NO: 44) |
| Human | cctgctgccctacacagg | agctctcatagtcctcggattg |
| Notch | (SEQ ID NO: 45) | (SEQ ID NO: 46) |
| 1 | | |
| Mouse | actcagtatgtggccctgcta | aacctgcctgccacctct |
| Lefty | (SEQ ID NO: 47) | (SEQ ID NO: 48) |
| 1 | | |
| Mouse | cacaagttggtccgtttcg | ggtacctcggggtcacaat |
| Lefty | (SEQ ID NO: 49) | (SEQ ID NO: 50) |
| 2 | | |
| Mouse | ccaaccatgcctacatcca | cacagcacgtggaaggaac |
| Nodal | (SEQ ID NO: 51) | (SEQ ID NO: 52) |
| Mouse | gactcatttcactagccagcag | cggcgattcttgaaccaa |
| Pitx2 | (SEQ ID NO: 53) | (SEQ ID NO: 54) |
| Mouse | ccaacaaggacatgcagaac | cagtctcatagctgccctca |
| Notch | (SEQ ID NO: 55) | (SEQ ID NO: 56) |
| 1 | | |

TABLE 3
Primers for regular PCR

| Gene | Primer forward | Primer reverse |
|---|---|---|
| Human Lefty1 | CTACAGGTGTCGGTGCAGAG(SEQ ID | AAGTCCCTCGATGGCTACACTA(SEQ ID |
| | NO: 57) | NO: 58) |
| Human Lefty2 | CGTCCATCACCCATCCTAAG (SEQ ID | CGTCCATCACCCATCCTAAG (SEQ ID |
| | NO: 59) | NO: 60) |
| Human Nodal | ATCATCTACCCCAAGCAGTACAAC | ACTGAGCCCTTCATTTACAGAGTG |
| | (SEQ ID NO: 61) | (SEQ ID NO: 62) |
| Human Pitx2 | GAGGACCCGTCTAAGAAGAAGC | TCAAGTTATTCAGGCTGTTGAGAC |
| | (SEQ ID NO: 63) | (SEQ ID NO: 64) |
| Human Notch1 | CAGAACTGTGAGGAAAATATCGAC | AGTTGGAGCCCTCGTTACAG (SEQ ID |
| | (SEQ ID NO: 65) | NO: 66) |
| Mouse Lefty1 | TCAGCCTGCCCAACATGA(SEQ ID NO: | TTCACATCTAGCAAAGCCAGT(SEQ ID |
| | 67) | NO: 68) |
| Mouse Lefty2 | CGT GAG GTC CCA GTA TGT GG | GTA GTC CTT GAG GTC CAG CG (SEQ |
| | (SEQ ID NO: 69) | ID NO: 70) |
| Mouse Nodal | GTG ACC GGA CAG AAC TGG AC | CTG TCT GGC AAA TGA TGT CG (SEQ |
| | (SEQ ID NO: 71) | ID NO: 72) |

### EXAMPLE 1

### Left-Right Gene Expression of Lefty2 and Pitx2 in Bipedal Mouse

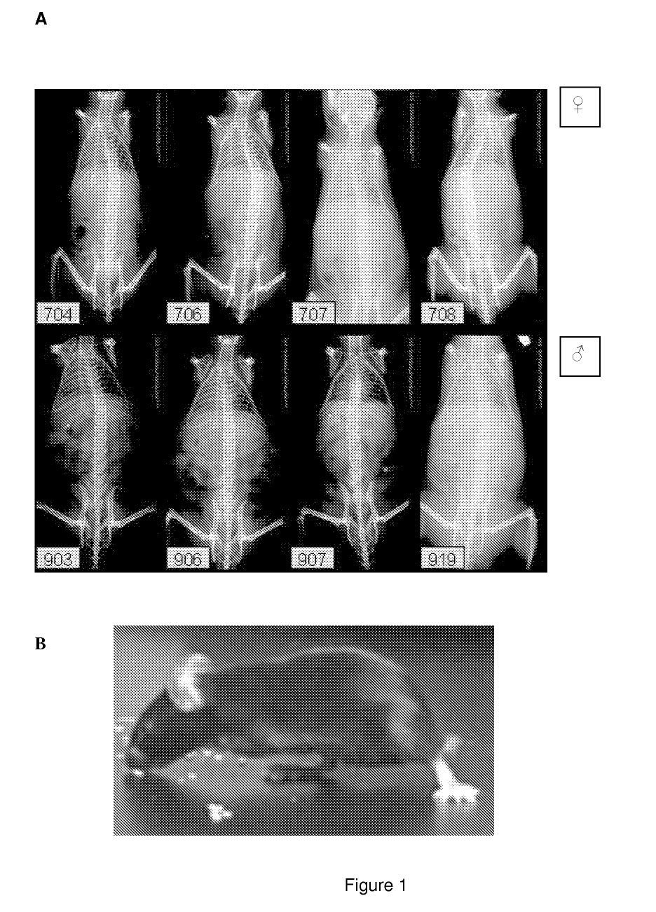

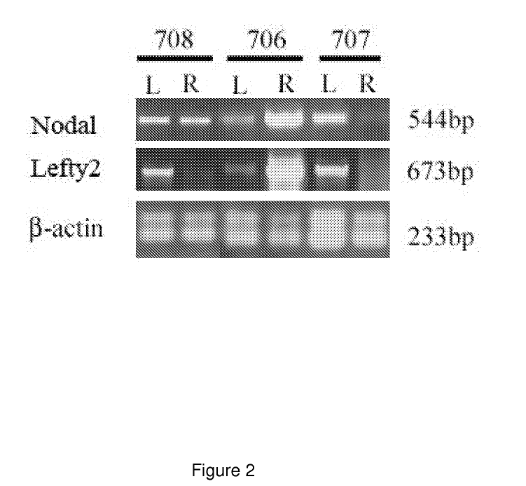

Bipedal mice were generated as explained above, 20 of which presented a scoliosis. FIG. 1A showing for instance mice #708 and #706 presenting left and right thoracic scoliosis, respectively and non scoliotic mouse #707. Biopsies of paraspinal muscles from the left and right sides were obtained as explained above. The left-right RNA expression of the genes Nodal, left-right determination factor 2 (Lefty2) and Pitx2 was analysed as explained above. As may be seen in FIGS. 1-2, these left-side restricted genes were expressed on the right side of the scoliotic bipedal mouse exhibiting a right thoracic curve (#706), while those genes were expressed on the left side of the non-scoliotic mouse. These changes occur at the apex of the curve. In the rare event of left thoracic scoliosis, these genes were still expressed on the left side although they were co-expressed with the same intensity on the right side or slightly overexpressed as shown in mouse #708 presenting a left thoracic scoliosis.

### EXAMPLE 2

### Left-Right Gene Expression of Lefty-2 and Pitx2 in AIS Patients

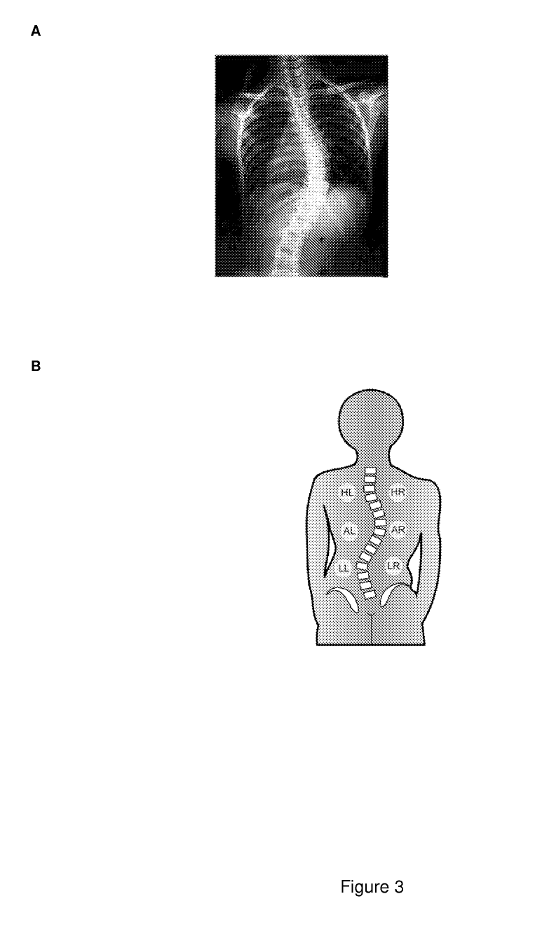

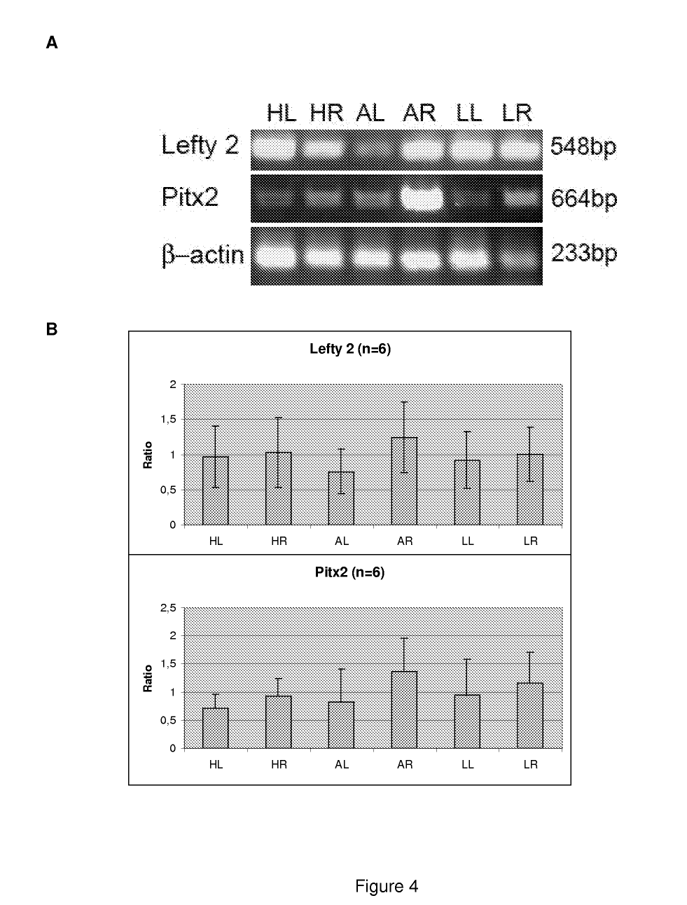

Biopsies of paraspinal muscles from the left and right sides right thoracic scoliotic patients (FIG. 3 A) were obtained as described above. Lefty-2 and Pitx2 were analyzed as presented above. As may be seen in FIG. 4, these left-sided genes were expressed on the right side at the apex of the curve and correlate with the side at which it bends.

### EXAMPLE 3

### Left-Right Gene Expression of Lefty1, Lefty2, Notch1, Nodal and Pitx2 in Paraspinal Muscles of AIS Patients

Paraspinal muscle biopsies were taken at the apex of the left and right sides of the vertebral column of AIS patients (n=6) and control subject (n=1). RNAs prepared from the biopsies were used to perform real-time quantitative PCR in order to assess changes in expression of LR-asymmetry genes Lefty1, Lefty2, Notch1, Pitx2 and Nodal as described above.

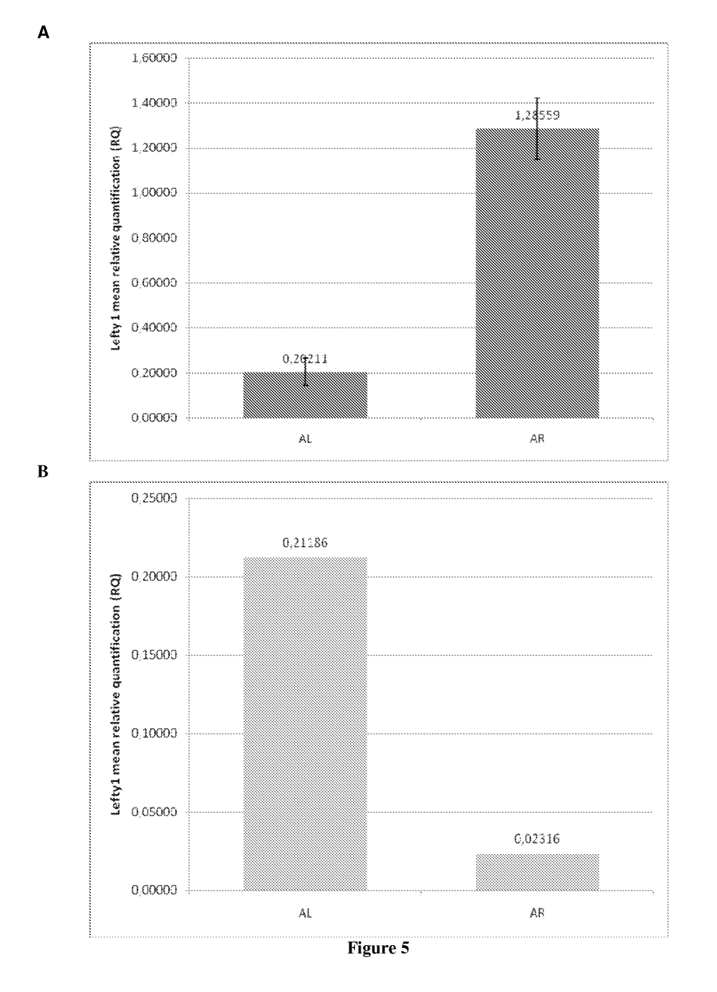

FIGS. 5-8 show mean relative quantifications of these genes expression on the left side (AL) compared to the right side (AR) in AIS patients (panel A) and control subjects (panel B).

As shown in the control subject (FIG. 5, panel B), Lefty1 is normally more highly expressed on the left side of the apex, while in AIS patients (FIG. 5, panel A), it is more highly expressed on the right side. This difference is statistically significant (Panel A: p=0,030).

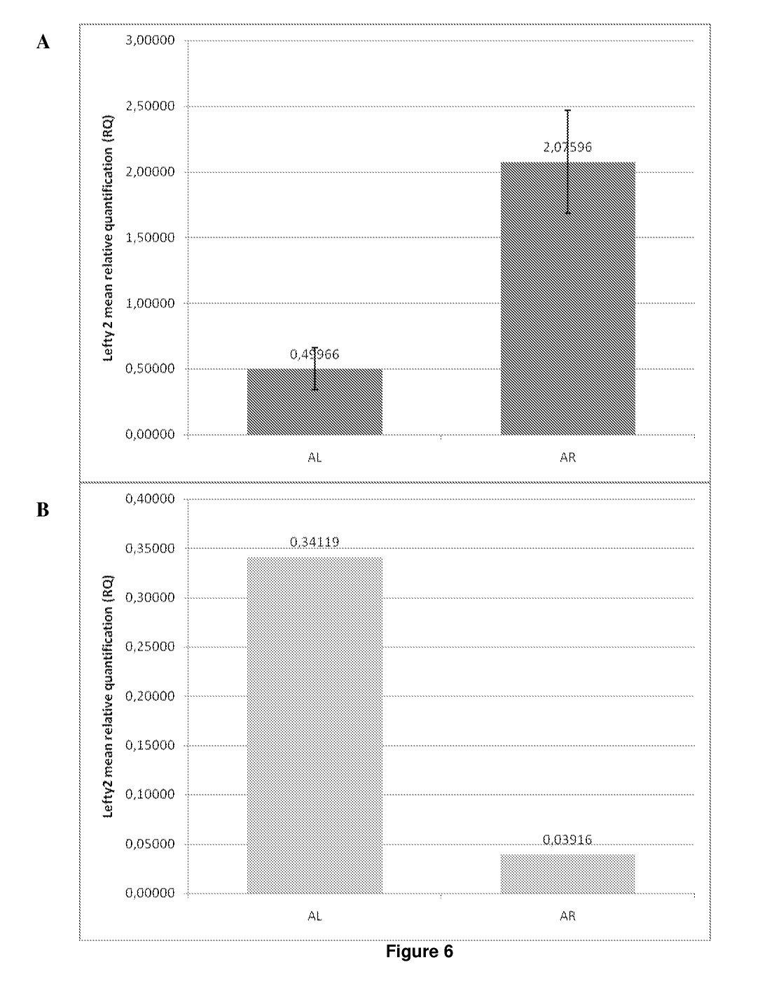

As shown in control subject (FIG. 6, panel B), Lefty2 is normally more highly expressed on the left side of the apex, while in AIS patients (FIG. 6, panel A), Lefty2 is more highly expressed on the right side. This difference is statistically significant (p=0.0019).

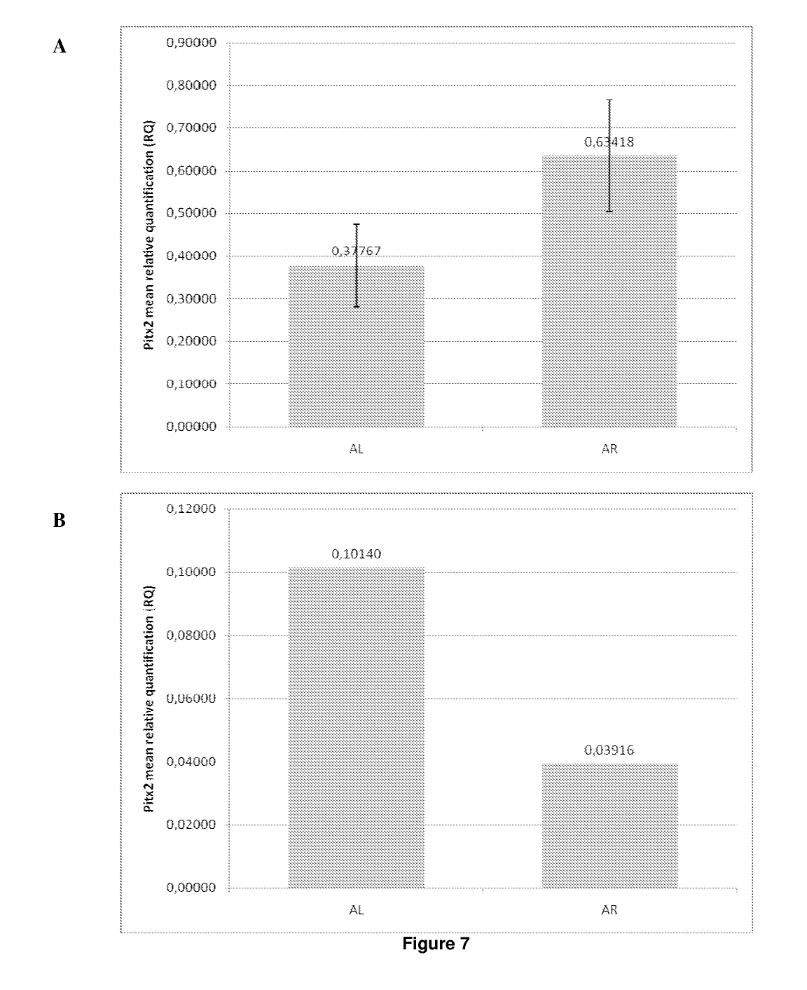

As shown in the control subject (FIG. 7, panel B), Pitx2 is normally more highly expressed on the left side of the apex, while in AIS patients (FIG. 7, panel A), it is more highly expressed on the right side.

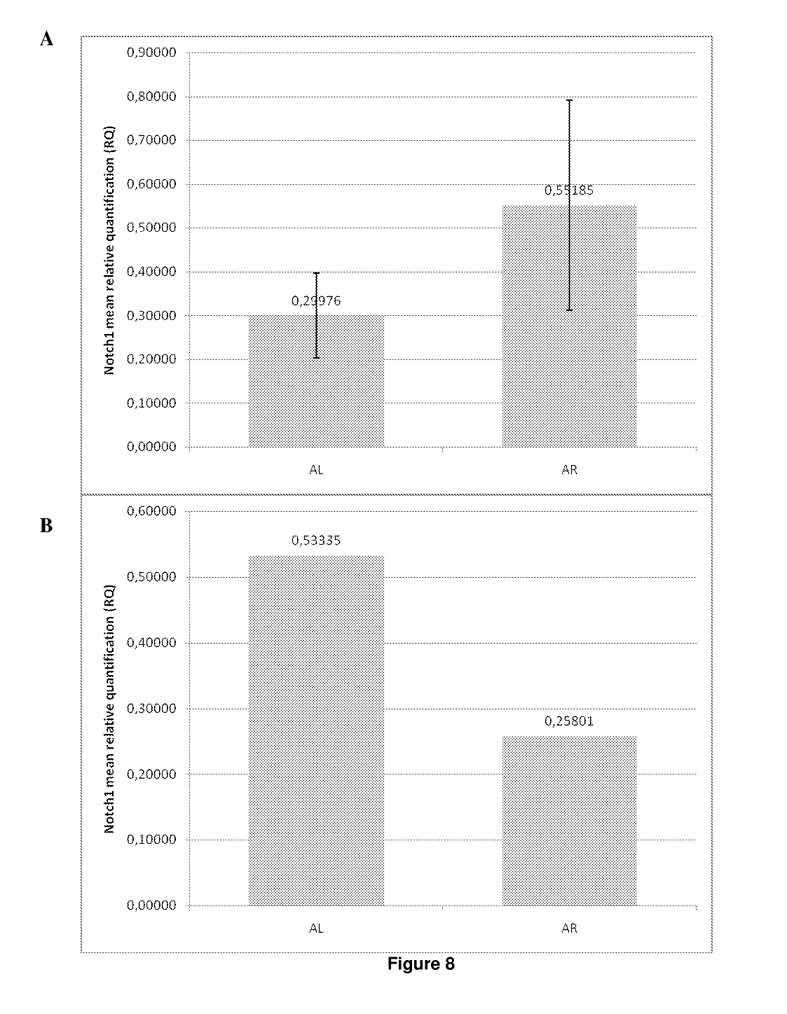

As shown in the control subject (FIG. 8, panel B), Notch1 is normally more highly expressed on the left side of the apex, while in AIS patients (FIG. 8, panel A), it is more highly expressed on the right side.

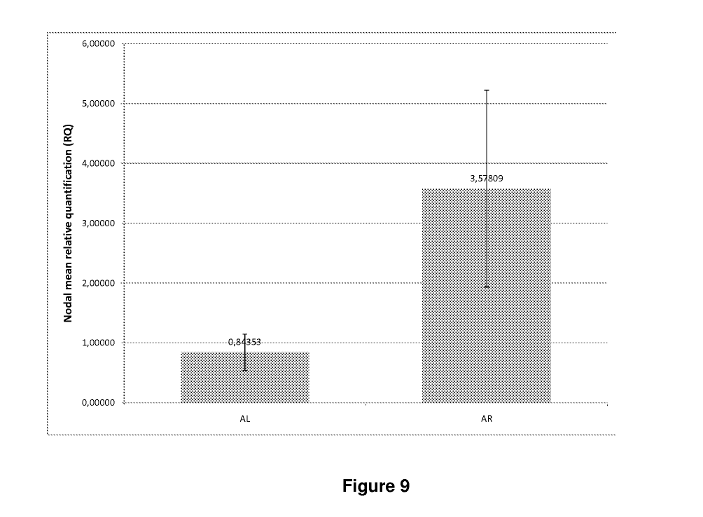

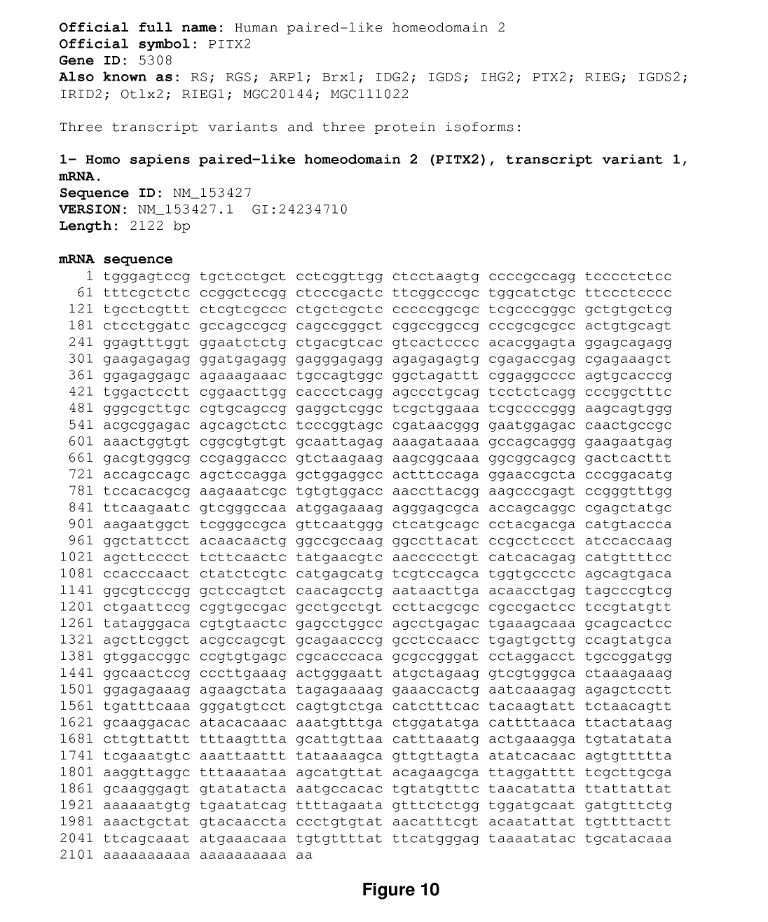

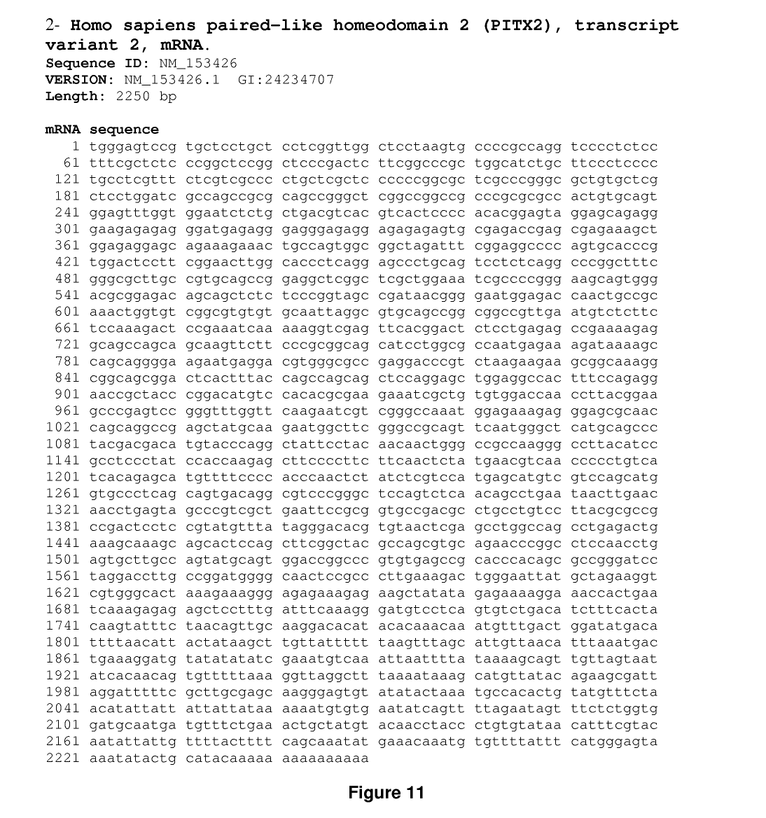

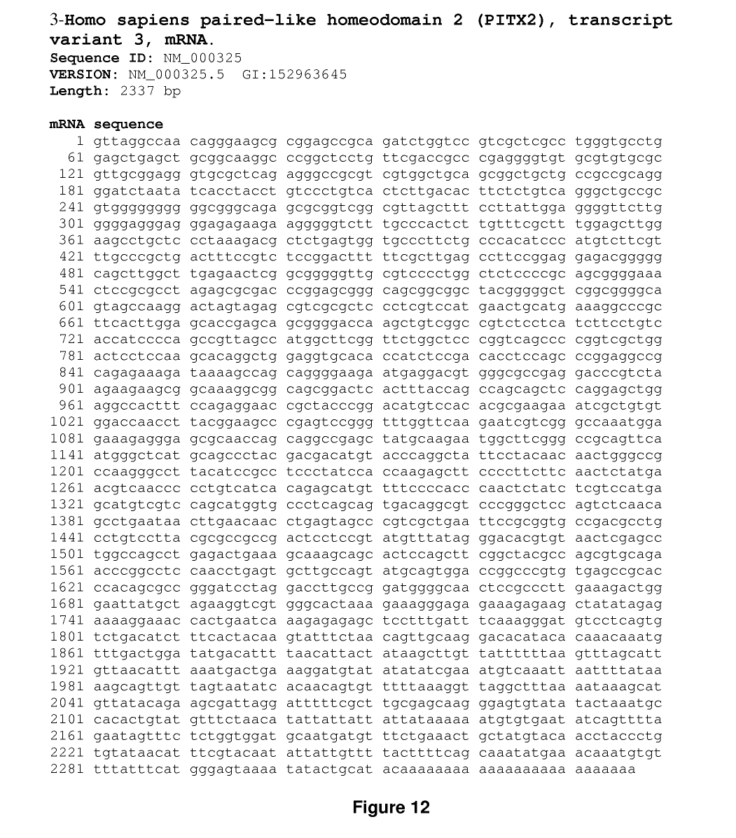

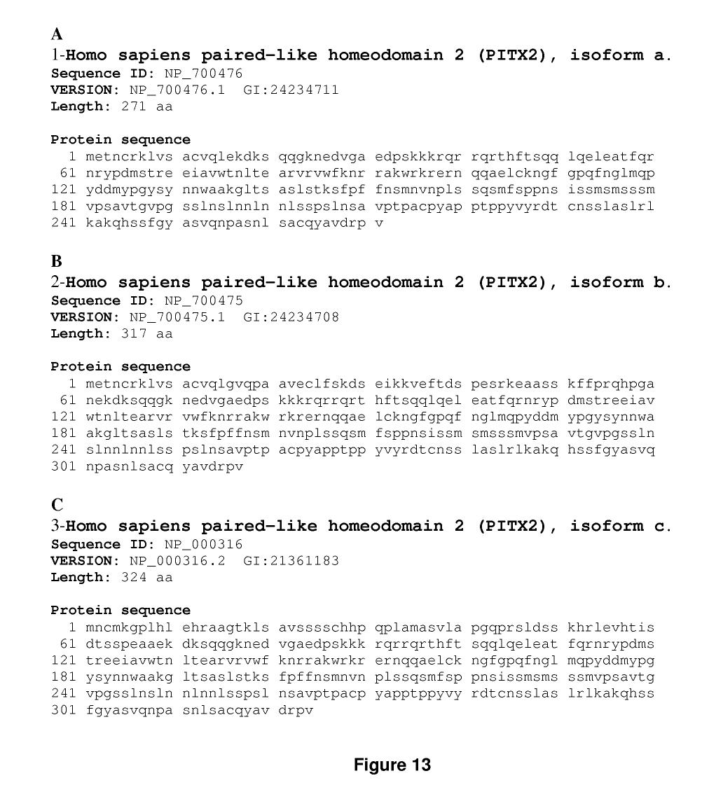

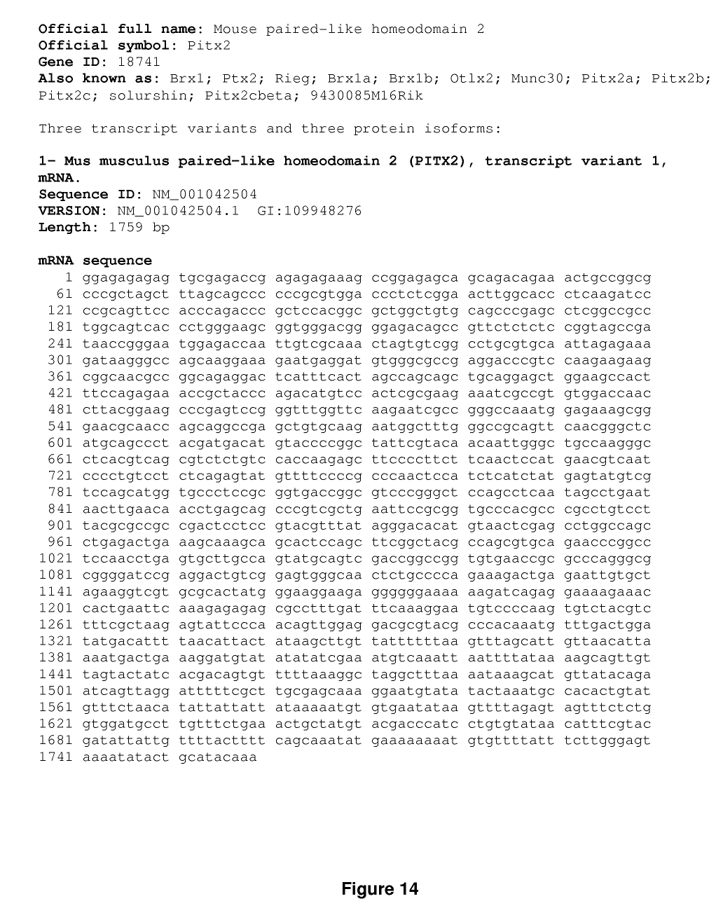

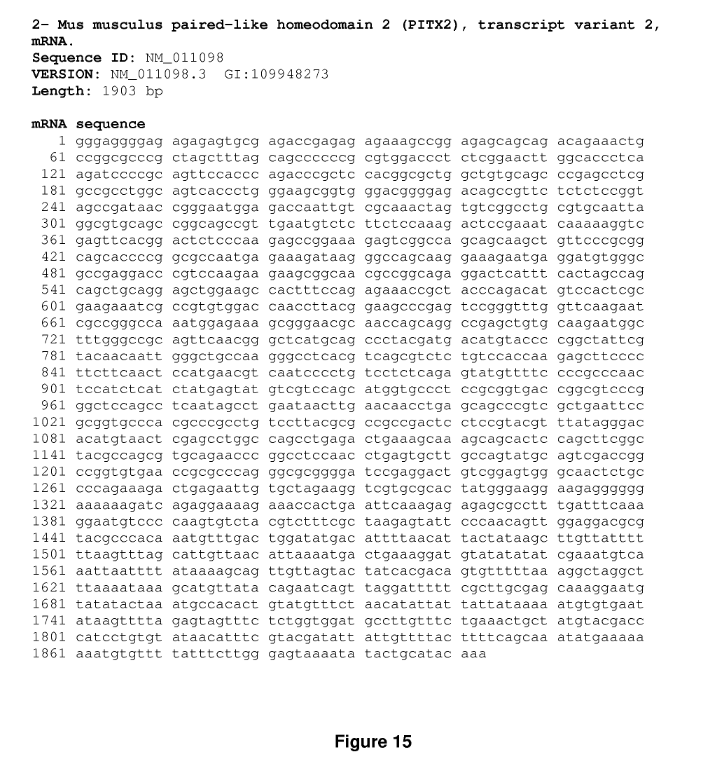

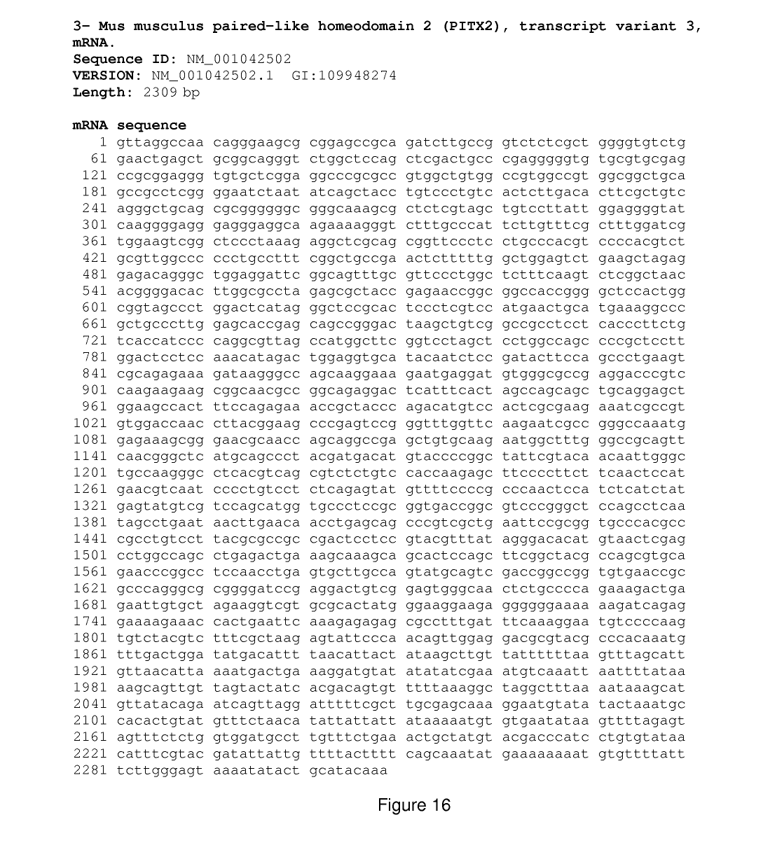

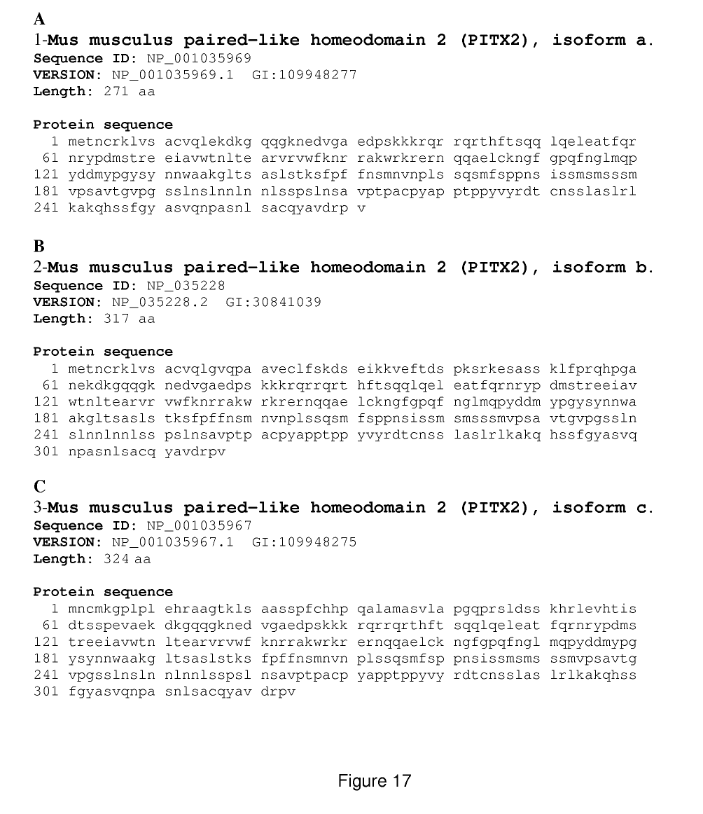

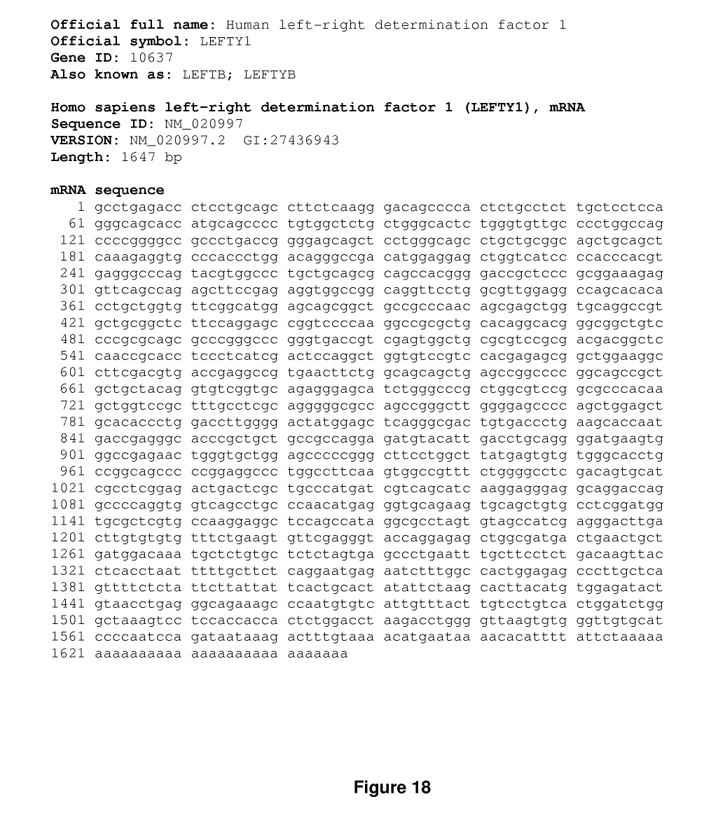

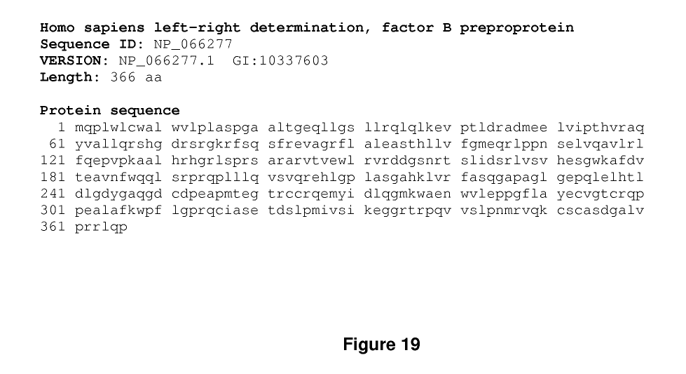

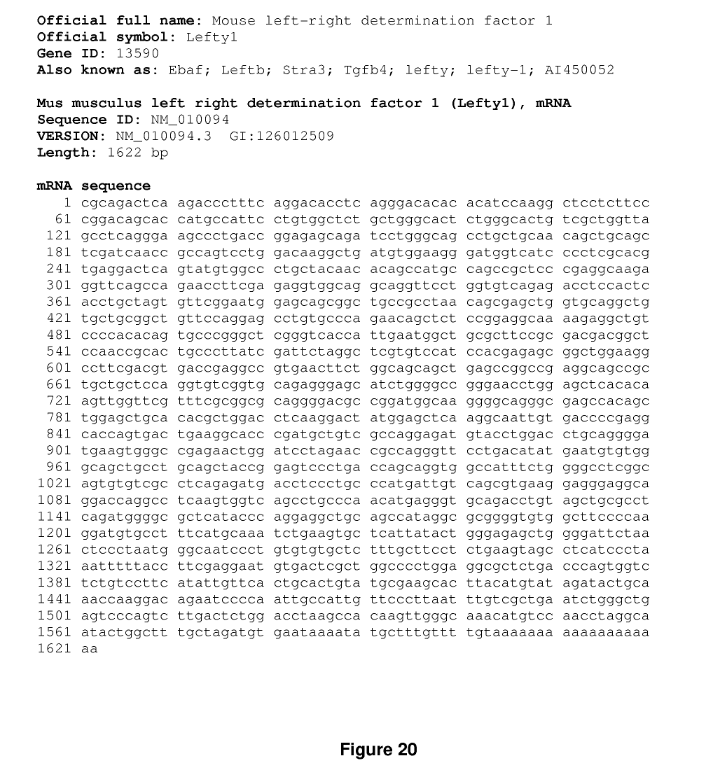

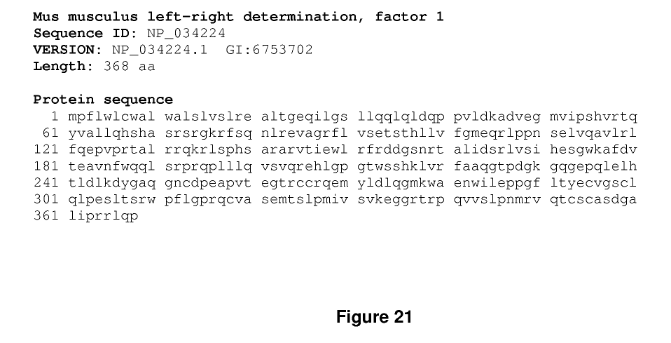

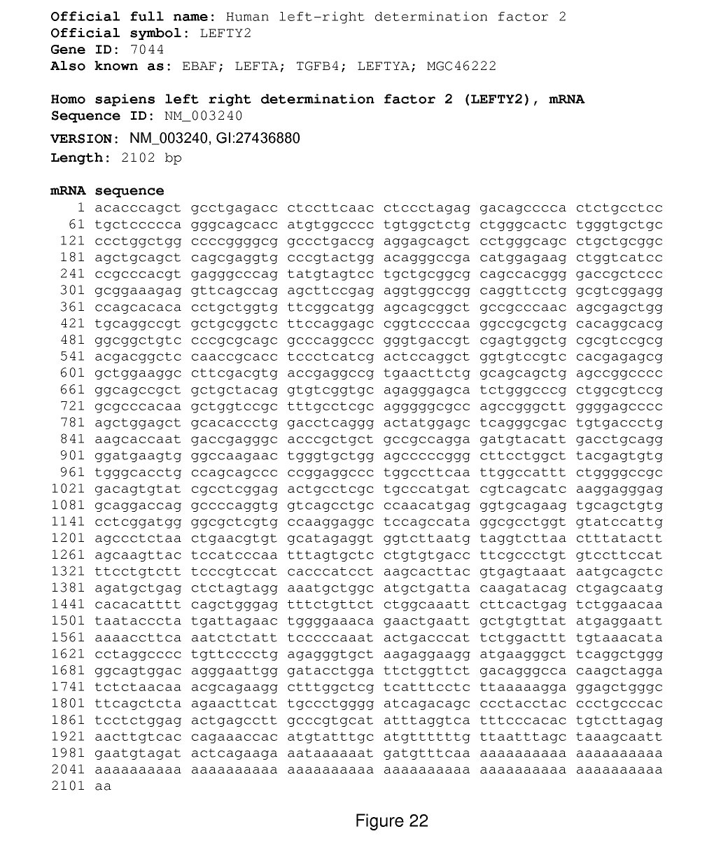

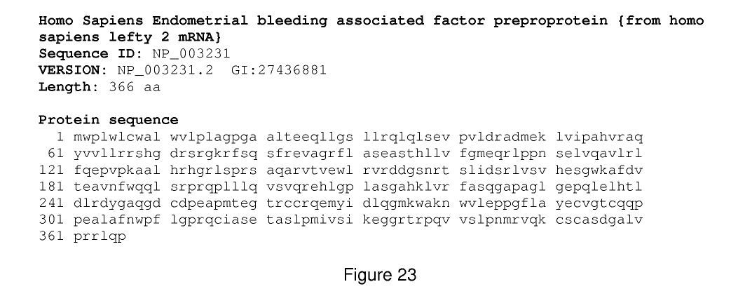

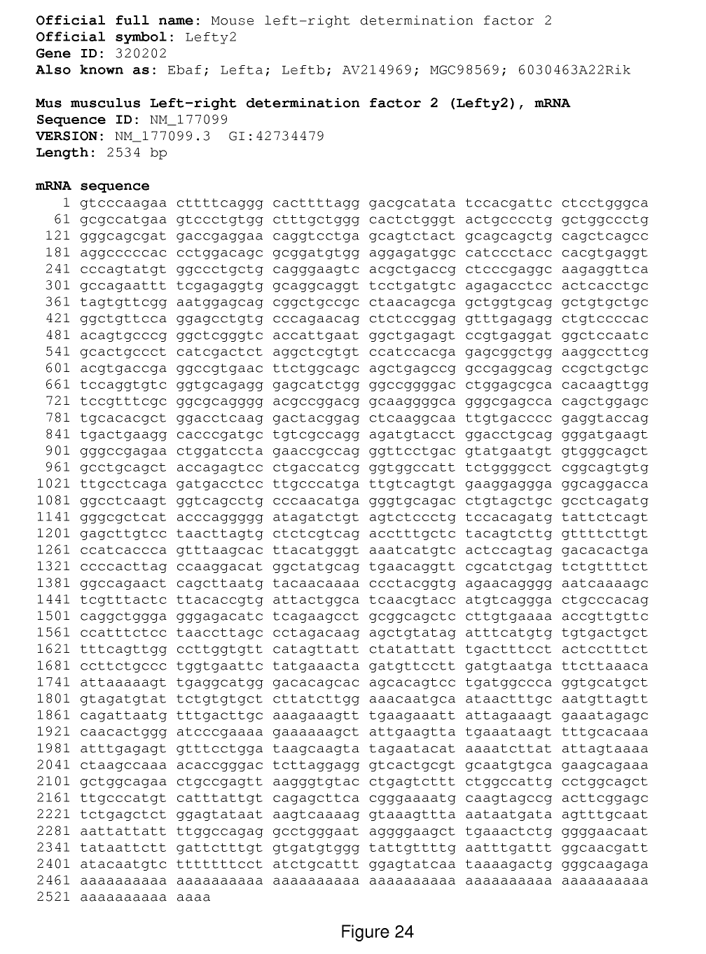

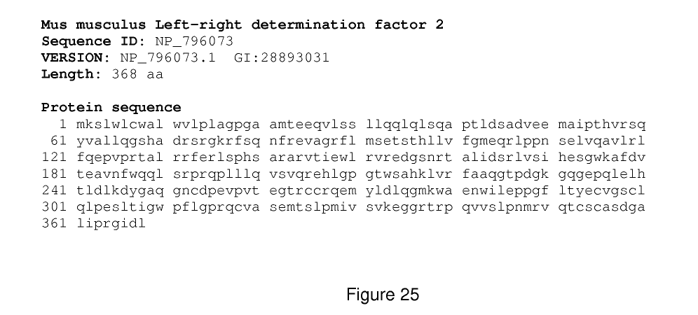

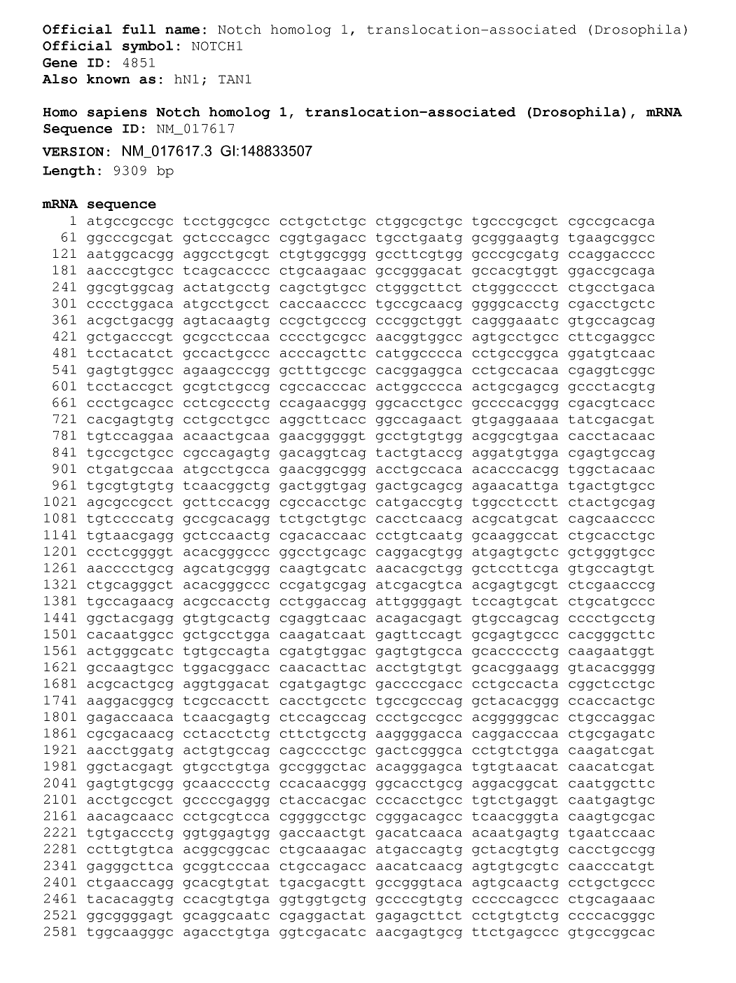

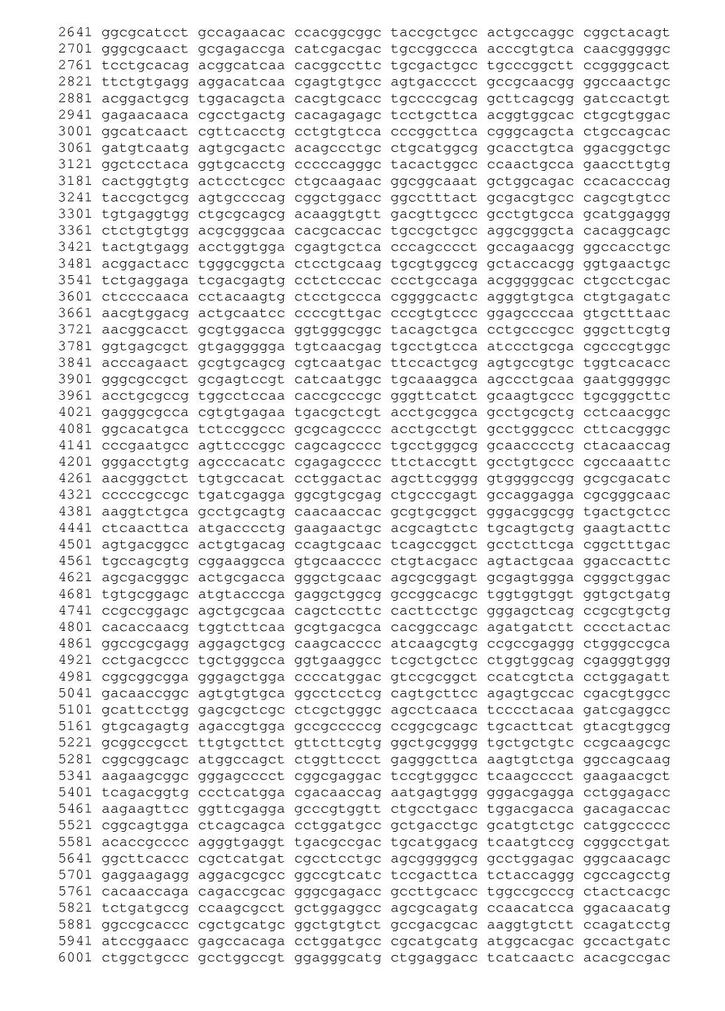

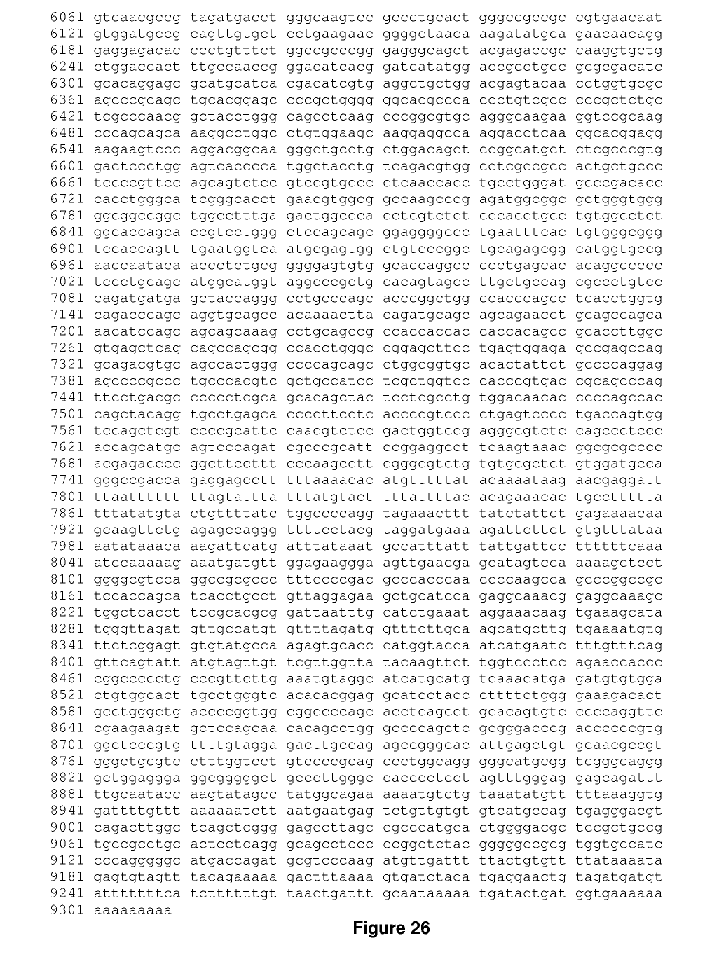

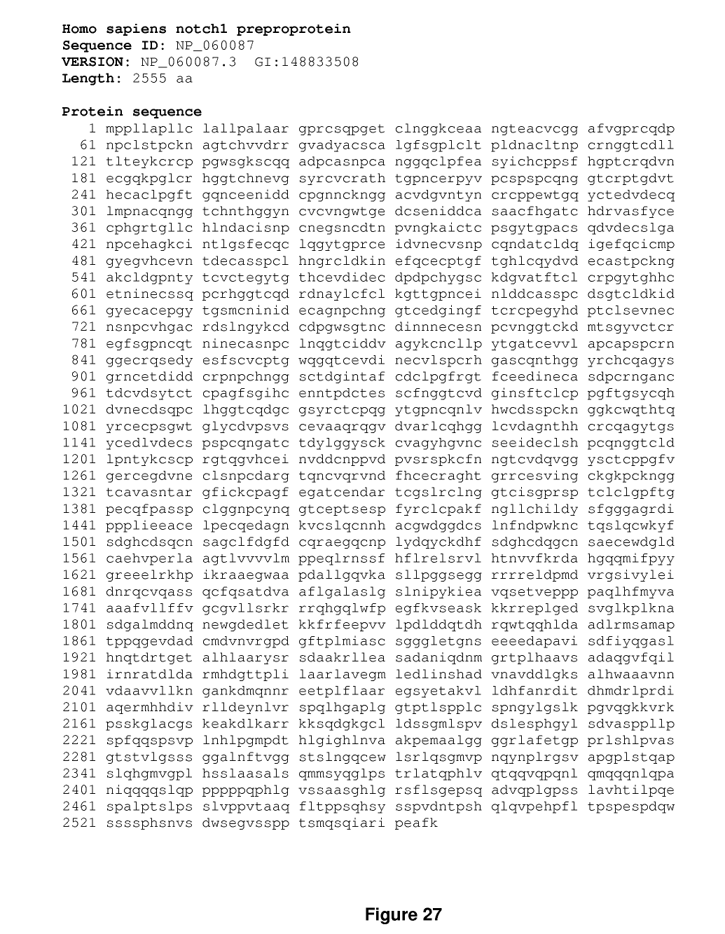

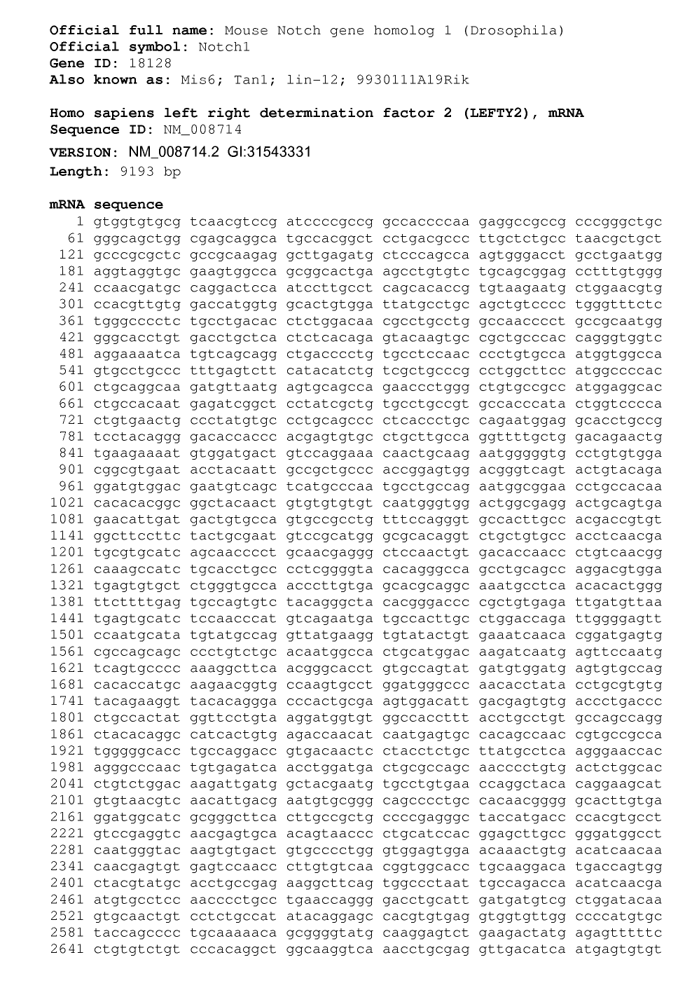

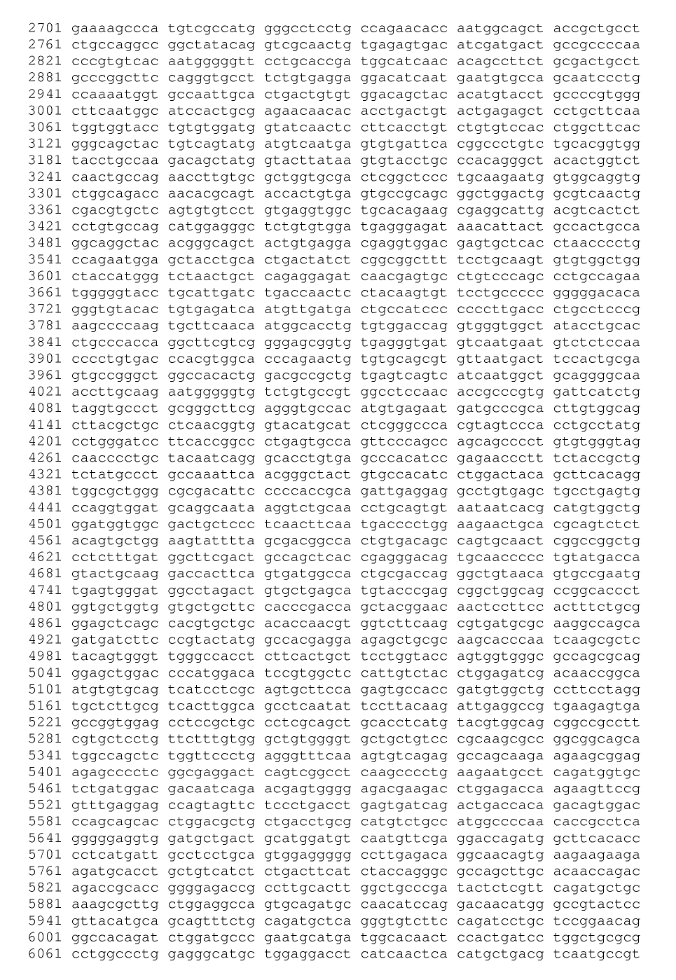

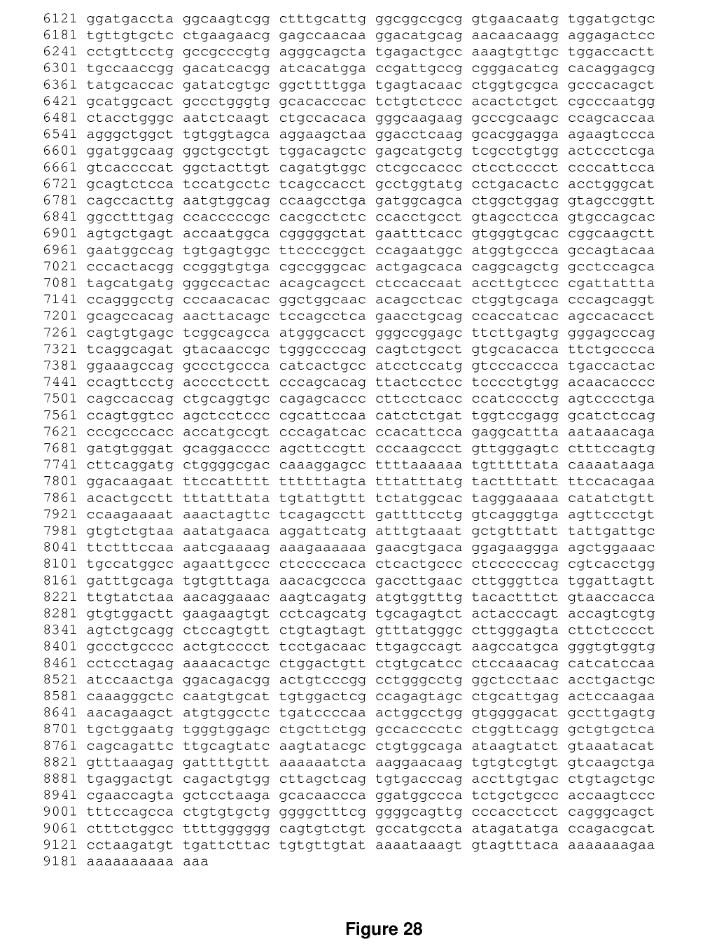

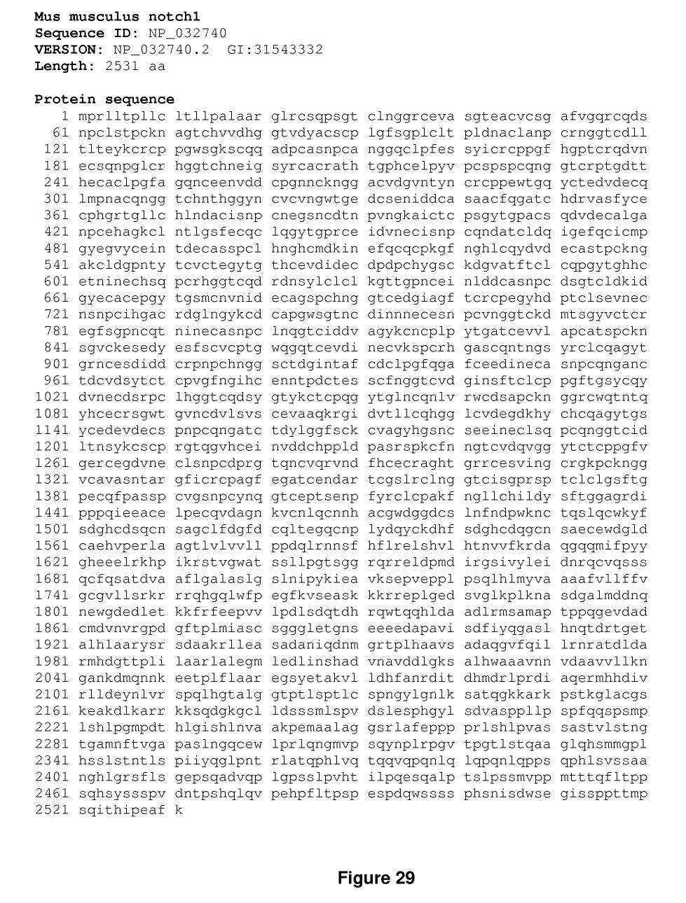

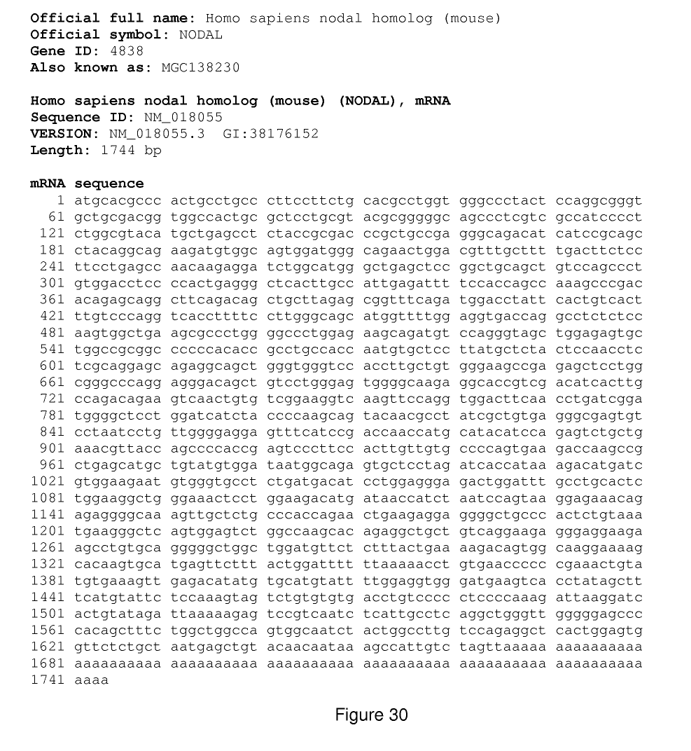

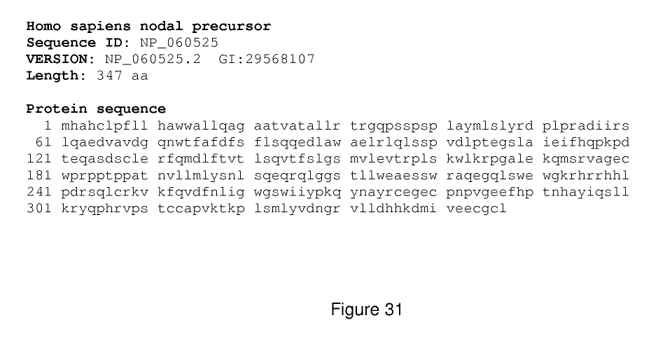

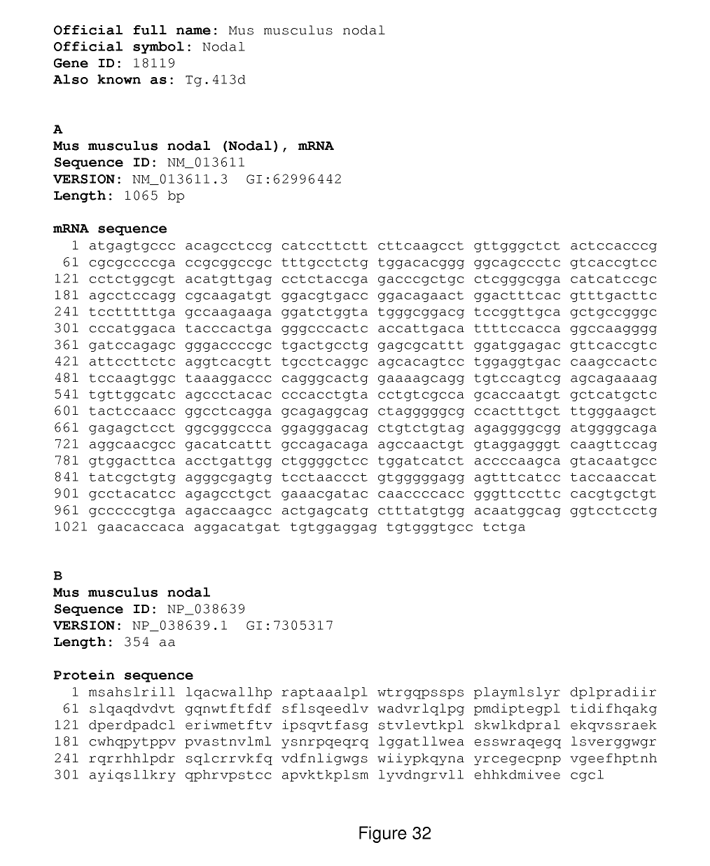

Nodal is normally more highly expressed on the left side of the apex, while in AIS patients (FIG. 9), it is more highly expressed on the right side.

### EXAMPLE 4

### Left-Right Gene Expression of Lefty1, Lefty2, Notch1 and Nodal and Pitx2 in Skin Fibroblasts of AIS Patients

The expression of genes shown to present differential expressions between AIS and control subjects in paraspinal samples are measured in skin fibroblasts by QPCR.

### EXAMPLE 5

### Gene Profile Expression of AIS Subjects

Gene profile expressions of AIS subjects in relevant samples (e.g., paraspinal muscle cell and skin fibroblasts samples) are then compared with those of control subjects. The expression of the genes presenting differential expressions between AIS and control subjects are measured by QPCR and further studied to determine if they belong to pathways to which either one of nodal, notch1, lefty1, Lefty2 or Pitx2 belongs. Expression of these genes are then knocked down in appropriate models to determine their effect on nodal, notch1, lefty1, Lefty2 or Pitx2's reverse asymmetrical expression. The sequences of these genes will be assessed to identify potential mutations. Expression of these genes is blocked in appropriate models to determine their effect on nodal, notch1, lefty1, Lefty2 or Pitx2's reverse asymmetrical expression. The sequences of these genes are assessed to identify potential mutations.

Although the present invention has been described herein above by way of specific embodiments thereof, it can be modified, without departing from the spirit and nature of the subject invention as defined in the appended claims.

### REFERENCES

1. Connor J M, Conner A N, Connor R A, Tolmie J L, Yeung B, Goudie D. Genetic aspects of early childhood scoliosis. Am J Med Genet. 1987; 27:419-424.

2. Machida M. Cause of idiopathic scoliosis. Spine. 1999; 24:2576-2583.

3. Drouin, J., Lanctôt, C., & Tremblay, J. J. La famille Ptx des facteurs de transcription à homéodomaine. Médecine/Sciences 14, 335-339 (1998).

4. Drouin, J., Lamolet, B., Lamonerie, T., Lanctot, C., & Tremblay, J. J. The PTX family of homeodomain transcription factors during pituitary developments. Mol. Cell Endocrinol. 140, 31-36 (1998).

5. Lanctot, C., Lamolet, B., & Drouin, J. The bicoid-related homeoprotein Ptx1 defines the most anterior domain of the embryo and differentiates posterior from anterior lateral mesoderm. Development 124, 2807-2817 (1997).

6. J L Rinn, C Bondre, H B Gladstone, P O Brown, and H Y Chang. Anatomic demarcation by positional variation in fibroblast gene expression programs. PLoS Genet, Jul. 1, 2006; 2(7): e119

7. Burwell et al. Stud Health Technol Inform. 2002.

8. von Gall C, Lewy A, Schomerus C et al. Transcription factor dynamics and neuroendocrine signalling in the mouse pineal gland: a comparative analysis of melatonin-deficient C57BL mice and melatonin-proficient C3H mice. Eur J Neurosci 2000; 12 (3) :964-972.

9. Aherrahrou Z, Axtner S B, Kaczmarek P M et al. A locus on chromosome 7 determines dramatic up-regulation of osteopontin in dystrophic cardiac calcification in mice. Am J Pathol 2004; 164(4):1379-1387.

10. Machida M, Dubousset J, Yamada T et al. Experimental scoliosis in melatonin-deficient C57BL/6J mice without pinealectomy. J Pineal Res 2006; 41(1):1-7.

1. A method of screening for a compound for treating or preventing adolescent idiopathic scoliosis (AIS), said method comprising:
(a) contacting a test compound with a paraspinal skin fibroblast or a paraspinal muscle cell sample from the right and/or left side of the spine of a subject; and
(b) determining at least one of Nodal, Notch1, Pitx2, Lefty1 and Lefty2's expression and/or activity in the cell sample;
wherein the test compound is selected as potentially useful in treating or preventing AIS if at least one of Nodal, Notch1, Pitx2, Lefty1 and Lefty2's expression and/or activity in the cell sample is different in the presence of the test compound as compared to in the absence thereof.

2. The method of claim 1, wherein the test compound is selected as potentially useful in treating AIS in a subject having a right thoracic curve when i) at least one of Nodal, Notch1, Pitx2, Lefty1 and Lefty2's expression in the cell sample from the left side of the spine is higher in the presence of the test compound as compared to in the absence thereof; or when ii) at least one of Nodal, Notch1, Pitx2, Lefty1 and Lefty2's expression in the cell sample from the right side of the spine is lower in the presence of the test compound as compared to in the absence thereof; or when iii) both i) and ii).

3. The method of claim 1, wherein the test compound is selected as potentially useful in treating AIS in a subject having a left thoracic curve when at least one of Nodal, Notch1, Pitx2, Lefty1 and Lefty2's expression in the cell sample from the left side of the spine is lower in the presence of the test compound as compared to in the absence thereof.

4. The method of claim 1, wherein the cell sample is from a subject having AIS.

5. The method of claim 1, wherein the cell sample is from a subject that is a likely candidate for developing AIS.

6. The method of claim 2, wherein the cell sample is from a subject exhibiting a right thoracic curve.

7. The method of claim 3, the cell sample is from a subject exhibiting a left thoracic curve.

8. A method of monitoring efficacy of an orthopaedic device in a subject having adolescent idiopathic scoliosis (AIS) comprising a) measuring expression of at least one of Nodal, Notch1, Pitx2, Lefty1 and Lefty2 in a sample of paraspinal muscle cells from at least one side of the apex of the main scoliosis curve of the subject before having installed the device on the subject; and b) repeating the measure of step a) after having installed the device on the subject, wherein a difference of expression of at least one of Nodal, Notch1, Pitx2, Lefty1 and Lefty2 between steps a) and b) provides an indication on the efficacy of the device.

9. The method of claim 8, wherein the main scoliosis curve is a right thoracic curve, and a lower expression of at least one of Nodal, Notch1, Pitx2, Lefty1 and Lefty2 in the sample from the right side of the apex in step b) as compared to that in step a) is an indication that the device is efficient.

10. The method of claim 8, wherein the main scoliosis curve is a right thoracic curve, and a higher expression of at least one of Nodal, Notch1, Pitx2, Lefty1 and Lefty2 in the sample from the left side of the apex in step b) as compared to that in step a) is an indication that the device is efficient.

11. A method of identifying at least one mutation directly or indirectly contributing to adolescent idiopathic scoliosis (AIS), comprising comparing the nucleotide sequence of a gene of at least one of Nodal, Notch 1, lefty1, Lefty2 and Pitx2 from a subject having AIS with that of the corresponding gene in a control subject, wherein the presence of a mutation in the gene of the subject is an indication that the mutation contributes to AIS.

12. The method of claim 11, wherein the nucleotide sequence is that of Nodal.

13. The method of claim 11, wherein the nucleotide sequence is that of Notch1.

14. The method of claim 11, wherein the nucleotide sequence is that of Lefty1.

15. The method of claim 11, wherein the nucleotide sequence is that of Lefty2.

16. The method of claim 11, wherein the nucleotide sequence is that of Pitx2.

17. A method for identifying at least one mutation directly or indirectly contributing to adolescent idiopathic scoliosis (AIS), comprising analyzing (i) the sequence of a gene whose product directly or indirectly modulates Nodal, Notch1, Pitx2, Lefty1 and/or Lefty2's expression, wherein the gene is from a subject having AIS, or (ii) analyzing the sequence of the gene's product,
wherein the presence of a mutation in the gene or in the gene's product is an indication that the mutation contributes to AIS in the subject.

18. The method of claim 17, wherein the method comprises detecting in the subject a mutation in a gene who's product directly or indirectly modulates Nodal, Notch1, Pitx2, Lefty1 and/or Lefty2's asymmetrical expression in paraspinal muscle cells.

19. A method for determining whether a test compound is useful for the prevention and/or treatment of adolescent idiopathic scoliosis, said method comprising:
(a) contacting said test compound with a cell comprising a first nucleic acid comprising a transcriptionally regulatory element normally associated with a Nodal, Notch1, Pitx2, Lefty1 or Lefty2 gene, operably linked to a second nucleic acid comprising a reporter gene encoding a reporter protein; and
(b) determining whether the reporter gene expression and/or reporter protein activity is modified in the presence of said test compound;
wherein said difference in reporter gene expression and/or reporter protein activity is indicative that said test compound may be used for prevention and/or treatment of adolescent idiopathic scoliosis.

20. The method of claim 8, wherein a) comprises measuring expression of Pitx2.

21. The method of claim 8, comprising in a) further measuring expression of:
(i) Nodal,
(ii) Notch1,
(iii) Pitx2,
(iv) Lefty1,
(v) Lefty2, or
(vi) any combination of (i) to (v),
in a sample of paraspinal muscle cells from at least one side of the apex of the main scoliosis curve of the subject before having installed the device on the subject; wherein a difference of expression of (i), (ii), (iii), (v) or (vi) between steps a) and b) provides an indication on the efficacy of the device.

22. The method of claim 20, comprising in a) further measuring expression of:
(i) Nodal,
(ii) Notch1,
(iii) Pitx2,
(iv) Lefty1,
(v) Lefty2, or
(vi) any combination of (i) to (v),
in a sample of paraspinal muscle cells from at least one side of the apex of the main scoliosis curve of the subject before having installed the device on the subject; wherein a difference of expression of (i), (ii), (iii), (v) or (vi) between steps a) and b) provides an indication on the efficacy of the device.


SEQUENCE LISTING (72 sequences)

SEQ ID NO: 1 - DNA, 2122 bp, homo sapiens
tgggagtccgtgctcctgctcctcggttggctcctaagtgccccgccaggtcccctctcctttcgctctcccggctccggctcccgactcttcggcccgctggcatctgcttccctcccctgcctcgtttctcgtcgcccctgctcgctccccccggcgctcgcccgggcgctgtgctcgctcctggatcgccagccgcgcagccgggctcggccggccgcccgcgcgccactgtgcagtggagtttggtggaatctctgctgacgtcacgtcactccccacacggagtaggagcagagggaagagagagggatgagagggagggagaggagagagagtgcgagaccgagcgagaaagctggagaggagcagaaagaaactgccagtggcggctagatttcggaggccccagtgcacccgtggactccttcggaacttggcaccctcaggagccctgcagtcctctcaggcccggctttcgggcgcttgccgtgcagccggaggctcggctcgctggaaatcgccccgggaagcagtgggacgcggagacagcagctctctcccggtagccgataacggggaatggagaccaactgccgcaaactggtgtcggcgtgtgtgcaattagagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaacccggcctccaacctgagtgcttgccagtatgcagtggaccggcccgtgtgagccgcacccacagcgccgggatcctaggaccttgccggatggggcaactccgcccttgaaagactgggaattatgctagaaggtcgtgggcactaaagaaagggagagaaagagaagctatatagagaaaaggaaaccactgaatcaaagagagagctcctttgatttcaaagggatgtcctcagtgtctgacatctttcactacaagtatttctaacagttgcaaggacacatacacaaacaaatgtttgactggatatgacattttaacattactataagcttgttattttttaagtttagcattgttaacatttaaatgactgaaaggatgtatatatatcgaaatgtcaaattaattttataaaagcagttgttagtaatatcacaacagtgtttttaaaggttaggctttaaaataaagcatgttatacagaagcgattaggatttttcgcttgcgagcaagggagtgtatatactaaatgccacactgtatgtttctaacatattattattattataaaaaatgtgtgaatatcagttttagaatagtttctctggtggatgcaatgatgtttctgaaactgctatgtacaacctaccctgtgtataacatttcgtacaatattattgttttacttttcagcaaatatgaaacaaatgtgttttatttcatgggagtaaaatatactgcatacaaaaaaaaaaaaaaaaaaaaaaaaa

SEQ ID NO: 2 - DNA, 2250 bp, homo sapiens
tgggagtccgtgctcctgctcctcggttggctcctaagtgccccgccaggtcccctctcctttcgctctcccggctccggctcccgactcttcggcccgctggcatctgcttccctcccctgcctcgtttctcgtcgcccctgctcgctccccccggcgctcgcccgggcgctgtgctcgctcctggatcgccagccgcgcagccgggctcggccggccgcccgcgcgccactgtgcagtggagtttggtggaatctctgctgacgtcacgtcactccccacacggagtaggagcagagggaagagagagggatgagagggagggagaggagagagagtgcgagaccgagcgagaaagctggagaggagcagaaagaaactgccagtggcggctagatttcggaggccccagtgcacccgtggactccttcggaacttggcaccctcaggagccctgcagtcctctcaggcccggctttcgggcgcttgccgtgcagccggaggctcggctcgctggaaatcgccccgggaagcagtgggacgcggagacagcagctctctcccggtagccgataacggggaatggagaccaactgccgcaaactggtgtcggcgtgtgtgcaattaggcgtgcagccggcggccgttgaatgtctcttctccaaagactccgaaatcaaaaaggtcgagttcacggactctcctgagagccgaaaagaggcagccagcagcaagttcttcccgcggcagcatcctggcgccaatgagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaacccggcctccaacctgagtgcttgccagtatgcagtggaccggcccgtgtgagccgcacccacagcgccgggatcctaggaccttgccggatggggcaactccgcccttgaaagactgggaattatgctagaaggtcgtgggcactaaagaaagggagagaaagagaagctatatagagaaaaggaaaccactgaatcaaagagagagctcctttgatttcaaagggatgtcctcagtgtctgacatctttcactacaagtatttctaacagttgcaaggacacatacacaaacaaatgtttgactggatatgacattttaacattactataagcttgttattttttaagtttagcattgttaacatttaaatgactgaaaggatgtatatatatcgaaatgtcaaattaattttataaaagcagttgttagtaatatcacaacagtgtttttaaaggttaggctttaaaataaagcatgttatacagaagcgattaggatttttcgcttgcgagcaagggagtgtatatactaaatgccacactgtatgtttctaacatattattattattataaaaaatgtgtgaatatcagttttagaatagtttctctggtggatgcaatgatgtttctgaaactgctatgtacaacctaccctgtgtataacatttcgtacaatattattgttttacttttcagcaaatatgaaacaaatgtgttttatttcatgggagtaaaatatactgcatacaaaaaaaaaaaaaaa

SEQ ID NO: 3 - DNA, 2337 bp, homo sapiens
gttaggccaacagggaagcgcggagccgcagatctggtccgtcgctcgcctgggtgcctggagctgagctgcggcaaggcccggctcctgttcgaccgcccgaggggtgtgcgtgtgcgcgttgcggagggtgcgctcagagggccgcgtcgtggctgcagcggctgctgccgccgcaggggatctaatatcacctacctgtccctgtcactcttgacacttctctgtcagggctgccgcgtggggggggggcgggcagagcgcggtcggcgttagctttccttattggaggggttcttgggggagggagggagagaagaagggggtctttgcccactcttgtttcgctttggagcttggaagcctgctccctaaagacgctctgagtggtgcccttctgcccacatcccatgtcttcgtttgcccgctgactttccgtctccggactttttcgcttgagccttccggaggagacgggggcagcttggcttgagaactcggcgggggttgcgtcccctggctctccccgcagcggggaaactccgcgcctagagcgcgacccggagcgggcagcggcggctacgggggctcggcggggcagtagccaaggactagtagagcgtcgcgctccctcgtccatgaactgcatgaaaggcccgcttcacttggagcaccgagcagcggggaccaagctgtcggccgtctcctcatcttcctgtcaccatccccagccgttagccatggcttcggttctggctcccggtcagccccggtcgctggactcctccaagcacaggctggaggtgcacaccatctccgacacctccagcccggaggccgcagagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaacccggcctccaacctgagtgcttgccagtatgcagtggaccggcccgtgtgagccgcacccacagcgccgggatcctaggaccttgccggatggggcaactccgcccttgaaagactgggaattatgctagaaggtcgtgggcactaaagaaagggagagaaagagaagctatatagagaaaaggaaaccactgaatcaaagagagagctcctttgatttcaaagggatgtcctcagtgtctgacatctttcactacaagtatttctaacagttgcaaggacacatacacaaacaaatgtttgactggatatgacattttaacattactataagcttgttattttttaagtttagcattgttaacatttaaatgactgaaaggatgtatatatatcgaaatgtcaaattaattttataaaagcagttgttagtaatatcacaacagtgtttttaaaggttaggctttaaaataaagcatgttatacagaagcgattaggatttttcgcttgcgagcaagggagtgtatatactaaatgccacactgtatgtttctaacatattattattattataaaaaatgtgtgaatatcagttttagaatagtttctctggtggatgcaatgatgtttctgaaactgctatgtacaacctaccctgtgtataacatttcgtacaatattattgttttacttttcagcaaatatgaaacaaatgtgttttatttcatgggagtaaaatatactgcatacaaaaaaaaaaaaaaaaaaaaaaaaa

SEQ ID NO: 4 - protein, 271 aa, homo sapiens
METNCRKLVSACVQLEKDKSQQGKNEDVGAEDPSKKKRQRRQRTHFTSQQLQELEATFQRNRYPDMSTREEIAVWTNLTEARVRVWFKNRRAKWRKRERNQQAELCKNGFGPQFNGLMQPYDDMYPGYSYNNWAAKGLTSASLSTKSFPFFNSMNVNPLSSQSMFSPPNSISSMSMSSSMVPSAVTGVPGSSLNSLNNLNNLSSPSLNSAVPTPACPYAPPTPPYVYRDTCNSSLASLRLKAKQHSSFGYASVQNPASNLSACQYAVDRPV

SEQ ID NO: 5 - protein, 317 aa, homo sapiens
METNCRKLVSACVQLGVQPAAVECLFSKDSEIKKVEFTDSPESRKEAASSKFFPRQHPGANEKDKSQQGKNEDVGAEDPSKKKRQRRQRTHFTSQQLQELEATFQRNRYPDMSTREEIAVWTNLTEARVRVWFKNRRAKWRKRERNQQAELCKNGFGPQFNGLMQPYDDMYPGYSYNNWAAKGLTSASLSTKSFPFFNSMNVNPLSSQSMFSPPNSISSMSMSSSMVPSAVTGVPGSSLNSLNNLNNLSSPSLNSAVPTPACPYAPPTPPYVYRDTCNSSLASLRLKAKQHSSFGYASVQNPASNLSACQYAVDRPV

SEQ ID NO: 6 - protein, 324 aa, homo sapiens
MNCMKGPLHLEHRAAGTKLSAVSSSSCHHPQPLAMASVLAPGQPRSLDSSKHRLEVHTISDTSSPEAAEKDKSQQGKNEDVGAEDPSKKKRQRRQRTHFTSQQLQELEATFQRNRYPDMSTREEIAVWTNLTEARVRVWFKNRRAKWRKRERNQQAELCKNGFGPQFNGLMQPYDDMYPGYSYNNWAAKGLTSASLSTKSFPFFNSMNVNPLSSQSMFSPPNSISSMSMSSSMVPSAVTGVPGSSLNSLNNLNNLSSPSLNSAVPTPACPYAPPTPPYVYRDTCNSSLASLRLKAKQHSSFGYASVQNPASNLSACQYAVDRPV

SEQ ID NO: 7 - DNA, 1759 bp, mus musculus
ggagagagagtgcgagaccgagagagaaagccggagagcagcagacagaaactgccggcgcccgctagctttagcagccccccgcgtggaccctctcggaacttggcaccctcaagatccccgcagttccacccagacccgctccacggcgctggctgtgcagcccgagcctcggccgcctggcagtcaccctgggaagcggtgggacggggagacagccgttctctctccggtagccgataaccgggaatggagaccaattgtcgcaaactagtgtcggcctgcgtgcaattagagaaagataagggccagcaaggaaagaatgaggatgtgggcgccgaggacccgtccaagaagaagcggcaacgccggcagaggactcatttcactagccagcagctgcaggagctggaagccactttccagagaaaccgctacccagacatgtccactcgcgaagaaatcgccgtgtggaccaaccttacggaagcccgagtccgggtttggttcaagaatcgccgggccaaatggagaaagcgggaacgcaaccagcaggccgagctgtgcaagaatggctttgggccgcagttcaacgggctcatgcagccctacgatgacatgtaccccggctattcgtacaacaattgggctgccaagggcctcacgtcagcgtctctgtccaccaagagcttccccttcttcaactccatgaacgtcaatcccctgtcctctcagagtatgttttccccgcccaactccatctcatctatgagtatgtcgtccagcatggtgccctccgcggtgaccggcgtcccgggctccagcctcaatagcctgaataacttgaacaacctgagcagcccgtcgctgaattccgcggtgcccacgcccgcctgtccttacgcgccgccgactcctccgtacgtttatagggacacatgtaactcgagcctggccagcctgagactgaaagcaaagcagcactccagcttcggctacgccagcgtgcagaacccggcctccaacctgagtgcttgccagtatgcagtcgaccggccggtgtgaaccgcgcccagggcgcggggatccgaggactgtcggagtgggcaactctgccccagaaagactgagaattgtgctagaaggtcgtgcgcactatgggaaggaagaggggggaaaaaagatcagaggaaaagaaaccactgaattcaaagagagagcgcctttgatttcaaaggaatgtccccaagtgtctacgtctttcgctaagagtattcccaacagttggaggacgcgtacgcccacaaatgtttgactggatatgacattttaacattactataagcttgttattttttaagtttagcattgttaacattaaaatgactgaaaggatgtatatatatcgaaatgtcaaattaattttataaaagcagttgttagtactatcacgacagtgtttttaaaggctaggctttaaaataaagcatgttatacagaatcagttaggatttttcgcttgcgagcaaaggaatgtatatactaaatgccacactgtatgtttctaacatattattattataaaaatgtgtgaatataagttttagagtagtttctctggtggatgccttgtttctgaaactgctatgtacgacccatcctgtgtataacatttcgtacgatattattgttttacttttcagcaaatatgaaaaaaaatgtgttttatttcttgggagtaaaatatactgcatacaaa

SEQ ID NO: 8 - DNA, 1903 bp, mus musculus
gggaggggagagagagtgcgagaccgagagagaaagccggagagcagcagacagaaactgccggcgcccgctagctttagcagccccccgcgtggaccctctcggaacttggcaccctcaagatccccgcagttccacccagacccgctccacggcgctggctgtgcagcccgagcctcggccgcctggcagtcaccctgggaagcggtgggacggggagacagccgttctctctccggtagccgataaccgggaatggagaccaattgtcgcaaactagtgtcggcctgcgtgcaattaggcgtgcagccggcagccgttgaatgtctcttctccaaagactccgaaatcaaaaaggtcgagttcacggactctcccaagagccggaaagagtcggccagcagcaagctgttcccgcggcagcaccccggcgccaatgagaaagataagggccagcaaggaaagaatgaggatgtgggcgccgaggacccgtccaagaagaagcggcaacgccggcagaggactcatttcactagccagcagctgcaggagctggaagccactttccagagaaaccgctacccagacatgtccactcgcgaagaaatcgccgtgtggaccaaccttacggaagcccgagtccgggtttggttcaagaatcgccgggccaaatggagaaagcgggaacgcaaccagcaggccgagctgtgcaagaatggctttgggccgcagttcaacgggctcatgcagccctacgatgacatgtaccccggctattcgtacaacaattgggctgccaagggcctcacgtcagcgtctctgtccaccaagagcttccccttcttcaactccatgaacgtcaatcccctgtcctctcagagtatgttttccccgcccaactccatctcatctatgagtatgtcgtccagcatggtgccctccgcggtgaccggcgtcccgggctccagcctcaatagcctgaataacttgaacaacctgagcagcccgtcgctgaattccgcggtgcccacgcccgcctgtccttacgcgccgccgactcctccgtacgtttatagggacacatgtaactcgagcctggccagcctgagactgaaagcaaagcagcactccagcttcggctacgccagcgtgcagaacccggcctccaacctgagtgcttgccagtatgcagtcgaccggccggtgtgaaccgcgcccagggcgcggggatccgaggactgtcggagtgggcaactctgccccagaaagactgagaattgtgctagaaggtcgtgcgcactatgggaaggaagaggggggaaaaaagatcagaggaaaagaaaccactgaattcaaagagagagcgcctttgatttcaaaggaatgtccccaagtgtctacgtctttcgctaagagtattcccaacagttggaggacgcgtacgcccacaaatgtttgactggatatgacattttaacattactataagcttgttattttttaagtttagcattgttaacattaaaatgactgaaaggatgtatatatatcgaaatgtcaaattaattttataaaagcagttgttagtactatcacgacagtgtttttaaaggctaggctttaaaataaagcatgttatacagaatcagttaggatttttcgcttgcgagcaaaggaatgtatatactaaatgccacactgtatgtttctaacatattattattataaaaatgtgtgaatataagttttagagtagtttctctggtggatgccttgtttctgaaactgctatgtacgacccatcctgtgtataacatttcgtacgatattattgttttacttttcagcaaatatgaaaaaaaatgtgttttatttcttgggagtaaaatatactgcatacaaa

SEQ ID NO: 9 - DNA, 2309 bp, mus musculus
gttaggccaacagggaagcgcggagccgcagatcttgccggtctctcgctggggtgtctggaactgagctgcggcagggtctggctccagctcgactgcccgagggggtgtgcgtgcgagccgcggagggtgtgctcggaggcccgcgccgtggctgtggccgtggccgtggcggctgcagccgcctcggggaatctaatatcagctacctgtccctgtcactcttgacacttcgctgtcagggctgcagcgcggggggcgggcaaagcgctctcgtagctgtccttattggaggggtatcaaggggagggagggaggcaagaaaagggtctttgcccattcttgtttcgctttggatcgtggaagtcggctccctaaagaggctcgcagcggttccctcctgcccacgtccccacgtctgcgttggcccccctgcctttcggctgccgaactctttttggctggagtctgaagctagaggagacagggctggaggattcggcagtttgcgttccctggctctttcaagtctcggctaacacggggacacttggcgcctagagcgctaccgagaaccggcggccaccggggctccactggcggtagccctggactcatagggctccgcactccctcgtccatgaactgcatgaaaggcccgctgcccttggagcaccgagcagccgggactaagctgtcggccgcctcctcacccttctgtcaccatccccaggcgttagccatggcttcggtcctagctcctggccagccccgctccttggactcctccaaacatagactggaggtgcatacaatctccgatacttccagccctgaagtcgcagagaaagataagggccagcaaggaaagaatgaggatgtgggcgccgaggacccgtccaagaagaagcggcaacgccggcagaggactcatttcactagccagcagctgcaggagctggaagccactttccagagaaaccgctacccagacatgtccactcgcgaagaaatcgccgtgtggaccaaccttacggaagcccgagtccgggtttggttcaagaatcgccgggccaaatggagaaagcgggaacgcaaccagcaggccgagctgtgcaagaatggctttgggccgcagttcaacgggctcatgcagccctacgatgacatgtaccccggctattcgtacaacaattgggctgccaagggcctcacgtcagcgtctctgtccaccaagagcttccccttcttcaactccatgaacgtcaatcccctgtcctctcagagtatgttttccccgcccaactccatctcatctatgagtatgtcgtccagcatggtgccctccgcggtgaccggcgtcccgggctccagcctcaatagcctgaataacttgaacaacctgagcagcccgtcgctgaattccgcggtgcccacgcccgcctgtccttacgcgccgccgactcctccgtacgtttatagggacacatgtaactcgagcctggccagcctgagactgaaagcaaagcagcactccagcttcggctacgccagcgtgcagaacccggcctccaacctgagtgcttgccagtatgcagtcgaccggccggtgtgaaccgcgcccagggcgcggggatccgaggactgtcggagtgggcaactctgccccagaaagactgagaattgtgctagaaggtcgtgcgcactatgggaaggaagaggggggaaaaaagatcagaggaaaagaaaccactgaattcaaagagagagcgcctttgatttcaaaggaatgtccccaagtgtctacgtctttcgctaagagtattcccaacagttggaggacgcgtacgcccacaaatgtttgactggatatgacattttaacattactataagcttgttattttttaagtttagcattgttaacattaaaatgactgaaaggatgtatatatatcgaaatgtcaaattaattttataaaagcagttgttagtactatcacgacagtgtttttaaaggctaggctttaaaataaagcatgttatacagaatcagttaggatttttcgcttgcgagcaaaggaatgtatatactaaatgccacactgtatgtttctaacatattattattataaaaatgtgtgaatataagttttagagtagtttctctggtggatgccttgtttctgaaactgctatgtacgacccatcctgtgtataacatttcgtacgatattattgttttacttttcagcaaatatgaaaaaaaatgtgttttatttcttgggagtaaaatatactgcatacaaa

SEQ ID NO: 10 - protein, 271 aa, mus musculus
METNCRKLVSACVQLEKDKGQQGKNEDVGAEDPSKKKRQRRQRTHFTSQQLQELEATFQRNRYPDMSTREEIAVWTNLTEARVRVWFKNRRAKWRKRERNQQAELCKNGFGPQFNGLMQPYDDMYPGYSYNNWAAKGLTSASLSTKSFPFFNSMNVNPLSSQSMFSPPNSISSMSMSSSMVPSAVTGVPGSSLNSLNNLNNLSSPSLNSAVPTPACPYAPPTPPYVYRDTCNSSLASLRLKAKQHSSFGYASVQNPASNLSACQYAVDRPV

SEQ ID NO: 11 - protein, 317 aa, mus musculus
METNCRKLVSACVQLGVQPAAVECLFSKDSEIKKVEFTDSPKSRKESASSKLFPRQHPGANEKDKGQQGKNEDVGAEDPSKKKRQRRQRTHFTSQQLQELEATFQRNRYPDMSTREEIAVWTNLTEARVRVWFKNRRAKWRKRERNQQAELCKNGFGPQFNGLMQPYDDMYPGYSYNNWAAKGLTSASLSTKSFPFFNSMNVNPLSSQSMFSPPNSISSMSMSSSMVPSAVTGVPGSSLNSLNNLNNLSSPSLNSAVPTPACPYAPPTPPYVYRDTCNSSLASLRLKAKQHSSFGYASVQNPASNLSACQYAVDRPV

SEQ ID NO: 12 - protein, 324 aa, mus musculus
MNCMKGPLPLEHRAAGTKLSAASSPFCHHPQALAMASVLAPGQPRSLDSSKHRLEVHTISDTSSPEVAEKDKGQQGKNEDVGAEDPSKKKRQRRQRTHFTSQQLQELEATFQRNRYPDMSTREEIAVWTNLTEARVRVWFKNRRAKWRKRERNQQAELCKNGFGPQFNGLMQPYDDMYPGYSYNNWAAKGLTSASLSTKSFPFFNSMNVNPLSSQSMFSPPNSISSMSMSSSMVPSAVTGVPGSSLNSLNNLNNLSSPSLNSAVPTPACPYAPPTPPYVYRDTCNSSLASLRLKAKQHSSFGYASVQNPASNLSACQYAVDRPV

SEQ ID NO: 13 - DNA, 1647 bp, homo sapiens
gcctgagaccctcctgcagccttctcaagggacagccccactctgcctcttgctcctccagggcagcaccatgcagcccctgtggctctgctgggcactctgggtgttgcccctggccagccccggggccgccctgaccggggagcagctcctgggcagcctgctgcggcagctgcagctcaaagaggtgcccaccctggacagggccgacatggaggagctggtcatccccacccacgtgagggcccagtacgtggccctgctgcagcgcagccacggggaccgctcccgcggaaagaggttcagccagagcttccgagaggtggccggcaggttcctggcgttggaggccagcacacacctgctggtgttcggcatggagcagcggctgccgcccaacagcgagctggtgcaggccgtgctgcggctcttccaggagccggtccccaaggccgcgctgcacaggcacgggcggctgtccccgcgcagcgcccgggcccgggtgaccgtcgagtggctgcgcgtccgcgacgacggctccaaccgcacctccctcatcgactccaggctggtgtccgtccacgagagcggctggaaggccttcgacgtgaccgaggccgtgaacttctggcagcagctgagccggccccggcagccgctgctgctacaggtgtcggtgcagagggagcatctgggcccgctggcgtccggcgcccacaagctggtccgctttgcctcgcagggggcgccagccgggcttggggagccccagctggagctgcacaccctggaccttggggactatggagctcagggcgactgtgaccctgaagcaccaatgaccgagggcacccgctgctgccgccaggagatgtacattgacctgcaggggatgaagtgggccgagaactgggtgctggagcccccgggcttcctggcttatgagtgtgtgggcacctgccggcagcccccggaggccctggccttcaagtggccgtttctggggcctcgacagtgcatcgcctcggagactgactcgctgcccatgatcgtcagcatcaaggagggaggcaggaccaggccccaggtggtcagcctgcccaacatgagggtgcagaagtgcagctgtgcctcggatggtgcgctcgtgccaaggaggctccagccataggcgcctagtgtagccatcgagggacttgacttgtgtgtgtttctgaagtgttcgagggtaccaggagagctggcgatgactgaactgctgatggacaaatgctctgtgctctctagtgagccctgaatttgcttcctctgacaagttacctcacctaatttttgcttctcaggaatgagaatctttggccactggagagcccttgctcagttttctctattcttattattcactgcactatattctaagcacttacatgtggagatactgtaacctgagggcagaaagcccaatgtgtcattgtttacttgtcctgtcactggatctgggctaaagtcctccaccaccactctggacctaagacctggggttaagtgtgggttgtgcatccccaatccagataataaagactttgtaaaacatgaataaaacacattttattctaaaaaaaaaaaaaaaaaaaaaaaaaaaaaaaa

SEQ ID NO: 14 - protein, 366 aa, homo sapiens
MQPLWLCWALWVLPLASPGAALTGEQLLGSLLRQLQLKEVPTLDRADMEELVIPTHVRAQYVALLQRSHGDRSRGKRFSQSFREVAGRFLALEASTHLLVFGMEQRLPPNSELVQAVLRLFQEPVPKAALHRHGRLSPRSARARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLGDYGAQGDCDPEAPMTEGTRCCRQEMYIDLQGMKWAENWVLEPPGFLAYECVGTCRQPPEALAFKWPFLGPRQCIASETDSLPMIVSIKEGGRTRPQVVSLPNMRVQKCSCASDGALVPRRLQP

SEQ ID NO: 15 - DNA, 1622 bp, mus musculus
cgcagactcaagaccctttcaggacacctcagggacacacacatccaaggctcctcttcccggacagcaccatgccattcctgtggctctgctgggcactctgggcactgtcgctggttagcctcagggaagccctgaccggagagcagatcctgggcagcctgctgcaacagctgcagctcgatcaaccgccagtcctggacaaggctgatgtggaagggatggtcatcccctcgcacgtgaggactcagtatgtggccctgctacaacacagccatgccagccgctcccgaggcaagaggttcagccagaaccttcgagaggtggcaggcaggttcctggtgtcagagacctccactcacctgctagtgttcggaatggagcagcggctgccgcctaacagcgagctggtgcaggctgtgctgcggctgttccaggagcctgtgcccagaacagctctccggaggcaaaagaggctgtccccacacagtgcccgggctcgggtcaccattgaatggctgcgcttccgcgacgacggctccaaccgcactgcccttatcgattctaggctcgtgtccatccacgagagcggctggaaggccttcgacgtgaccgaggccgtgaacttctggcagcagctgagccggccgaggcagccgctgctgctccaggtgtcggtgcagagggagcatctggggccgggaacctggagctcacacaagttggttcgtttcgcggcgcaggggacgccggatggcaaggggcagggcgagccacagctggagctgcacacgctggacctcaaggactatggagctcaaggcaattgtgaccccgaggcaccagtgactgaaggcacccgatgctgtcgccaggagatgtacctggacctgcaggggatgaagtgggccgagaactggatcctagaaccgccagggttcctgacatatgaatgtgtgggcagctgcctgcagctaccggagtccctgaccagcaggtggccatttctggggcctcggcagtgtgtcgcctcagagatgacctccctgcccatgattgtcagcgtgaaggagggaggcaggaccaggcctcaagtggtcagcctgcccaacatgagggtgcagacctgtagctgcgcctcagatggggcgctcatacccaggaggctgcagccataggcgcggggtgtggcttccccaaggatgtgcctttcatgcaaatctgaagtgctcattatactgggagagctggggattctaactccctaatgggcaatccctgtgtgtgctctttgcttcctctgaagtagcctcatccctaaatttttaccttcgaggaatgtgactcgctggcccctggaggcgctctgacccagtggtctctgtccttcatattgttcactgcactgtatgcgaagcacttacatgtatagatactgcaaaccaaggacagaatccccaattgccattgttcccttaatttgtcgctgaatctgggctgagtcccagtcttgactctggacctaagccacaagttgggcaaacatgtccaacctaggcaatactggctttgctagatgtgaataaaatatgctttgttttgtaaaaaaaaaaaaaaaaaaa

SEQ ID NO: 16 - protein, 368 aa, mus musculus
MPFLWLCWALWALSLVSLREALTGEQILGSLLQQLQLDQPPVLDKADVEGMVIPSHVRTQYVALLQHSHASRSRGKRFSQNLREVAGRFLVSETSTHLLVFGMEQRLPPNSELVQAVLRLFQEPVPRTALRRQKRLSPHSARARVTIEWLRFRDDGSNRTALIDSRLVSIHESGWKAFDVTEAVNFWQQLSRPRQPLLLQVSVQREHLGPGTWSSHKLVRFAAQGTPDGKGQGEPQLELHTLDLKDYGAQGNCDPEAPVTEGTRCCRQEMYLDLQGMKWAENWILEPPGFLTYECVGSCLQLPESLTSRWPFLGPRQCVASEMTSLPMIVSVKEGGRTRPQVVSLPNMRVQTCSCASDGALIPRRLQP

SEQ ID NO: 17 - DNA, 2102 bp, homo sapiens
acacccagctgcctgagaccctccttcaacctccctagaggacagccccactctgcctcctgctcccccagggcagcaccatgtggcccctgtggctctgctgggcactctgggtgctgcccctggctggccccggggcggccctgaccgaggagcagctcctgggcagcctgctgcggcagctgcagctcagcgaggtgcccgtactggacagggccgacatggagaagctggtcatccccgcccacgtgagggcccagtatgtagtcctgctgcggcgcagccacggggaccgctcccgcggaaagaggttcagccagagcttccgagaggtggccggcaggttcctggcgtcggaggccagcacacacctgctggtgttcggcatggagcagcggctgccgcccaacagcgagctggtgcaggccgtgctgcggctcttccaggagccggtccccaaggccgcgctgcacaggcacgggcggctgtccccgcgcagcgcccaggcccgggtgaccgtcgagtggctgcgcgtccgcgacgacggctccaaccgcacctccctcatcgactccaggctggtgtccgtccacgagagcggctggaaggccttcgacgtgaccgaggccgtgaacttctggcagcagctgagccggccccggcagccgctgctgctacaggtgtcggtgcagagggagcatctgggcccgctggcgtccggcgcccacaagctggtccgctttgcctcgcagggggcgccagccgggcttggggagccccagctggagctgcacaccctggacctcagggactatggagctcagggcgactgtgaccctgaagcaccaatgaccgagggcacccgctgctgccgccaggagatgtacattgacctgcaggggatgaagtgggccaagaactgggtgctggagcccccgggcttcctggcttacgagtgtgtgggcacctgccagcagcccccggaggccctggccttcaattggccatttctggggccgcgacagtgtatcgcctcggagactgcctcgctgcccatgatcgtcagcatcaaggagggaggcaggaccaggccccaggtggtcagcctgcccaacatgagggtgcagaagtgcagctgtgcctcggatggggcgctcgtgccaaggaggctccagccataggcgcctggtgtatccattgagccctctaactgaacgtgtgcatagaggtggtcttaatgtaggtcttaactttatacttagcaagttactccatcccaatttagtgctcctgtgtgaccttcgccctgtgtccttccatttcctgtctttcccgtccatcacccatcctaagcacttacgtgagtaaataatgcagctcagatgctgagctctagtaggaaatgctggcatgctgattacaagatacagctgagcaatgcacacattttcagctgggagtttctgttctctggcaaattcttcactgagtctggaacaataataccctatgattagaactggggaaacagaactgaattgctgtgttatatgaggaattaaaaccttcaaatctctatttcccccaaatactgacccattctggacttttgtaaacatacctaggcccctgttcccctgagagggtgctaagaggaaggatgaagggcttcaggctgggggcagtggacagggaattgggatacctggattctggttctgacagggccacaagctaggatctctaacaaacgcagaaggctttggctcgtcatttcctcttaaaaaggaggagctgggcttcagctctaagaacttcattgccctggggatcagacagcccctacctacccctgcccactcctctggagactgagccttgcccgtgcatatttaggtcatttcccacactgtcttagagaacttgtcaccagaaaccacatgtatttgcatgttttttgttaatttagctaaagcaattgaatgtagatactcagaagaaataaaaaatgatgtttcaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - protein, 366 aa, homo sapiens
MWPLWLCWALWVLPLAGPGAALTEEQLLGSLLRQLQLSEVPVLDRADMEKLVIPAHVRAQYVVLLRRSHGDRSRGKRFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQP

SEQ ID NO: 19 - DNA, 2534 bp, mus musculus
gtcccaagaacttttcagggcacttttagggacgcatatatccacgattcctcctgggcagcgccatgaagtccctgtggctttgctgggcactctgggtactgcccctggctggccctggggcagcgatgaccgaggaacaggtcctgagcagtctactgcagcagctgcagctcagccaggcccccaccctggacagcgcggatgtggaggagatggccatccctacccacgtgaggtcccagtatgtggccctgctgcagggaagtcacgctgaccgctcccgaggcaagaggttcagccagaattttcgagaggtggcaggcaggttcctgatgtcagagacctccactcacctgctagtgttcggaatggagcagcggctgccgcctaacagcgagctggtgcaggctgtgctgcggctgttccaggagcctgtgcccagaacagctctccggaggtttgagaggctgtccccacacagtgcccgggctcgggtcaccattgaatggctgagagtccgtgaggatggctccaatcgcactgccctcatcgactctaggctcgtgtccatccacgagagcggctggaaggccttcgacgtgaccgaggccgtgaacttctggcagcagctgagccggccgaggcagccgctgctgctccaggtgtcggtgcagagggagcatctggggccggggacctggagcgcacacaagttggtccgtttcgcggcgcaggggacgccggacggcaaggggcagggcgagccacagctggagctgcacacgctggacctcaaggactacggagctcaaggcaattgtgaccccgaggtaccagtgactgaaggcacccgatgctgtcgccaggagatgtacctggacctgcaggggatgaagtgggccgagaactggatcctagaaccgccagggttcctgacgtatgaatgtgtgggcagctgcctgcagctaccagagtccctgaccatcgggtggccatttctggggcctcggcagtgtgttgcctcagagatgacctccttgcccatgattgtcagtgtgaaggagggaggcaggaccaggcctcaagtggtcagcctgcccaacatgagggtgcagacctgtagctgcgcctcagatggggcgctcatacccagggggatagatctgtagtctccctgtccacagatgtattctcagtgagcttgtcctaacttagtgctctcgtcagacctttgctctacagtcttggttttcttgtccatcacccagtttaagcacttacatgggtaaatcatgtcactccagtaggacacactgaccccacttagccaaggacatggctatgcagtgaacaggttcgcatctgagtctgttttctggccagaactcagcttaatgtacaacaaaaccctacggtgagaacaggggaatcaaaagctcgtttactcttacaccgtgattactggcatcaacgtaccatgtcagggactgcccacagcaggctgggagggagacatctcagaagcctgcggcagctccttgtgaaaaaccgttgttcccatttctcctaaccttagccctagacaagagctgtatagatttcatgtgtgtgactgcttttcagttggccttggtgttcatagttattctatattatttgactttcctactcctttctccttctgccctggtgaattctatgaaactagatgttccttgatgtaatgattcttaaacaattaaaaagttgaggcatgggacacagcacagcacagtcctgatggcccaggtgcatgctgtagatgtattctgtgtgctcttatcttggaaacaatgcaataactttgcaatgttagttcagattaatgtttgacttgcaaagaaagtttgaagaaattattagaaagtgaaatagagccaacactgggatcccgaaaagaaaaaagctattgaagttatgaaataagttttgcacaaaatttgagagtgtttcctggataagcaagtatagaatacataaaatcttatattagtaaaactaagccaaaacaccgggactcttaggagggtcactgcgtgcaatgtgcagaagcagaaagctggcagaactgccgagttaagggtgtacctgagtctttctggccattgcctggcagctttgcccatgtcatttattgtcagagcttcacgggaaaatgcaagtagccgacttcggagctctgagctctggagtataataagtcaaaaggtaaagtttaaataatgataagtttgcaataattattattttggccagaggcctgggaataggggaagcttgaaactctgggggaacaattataattcttgattctttgtgtgatgtgggtattgttttgaatttgatttggcaacgattatacaatgtctttttttcctatctgcatttggagtatcaataaaagactggggcaagagaaaaaaaaaaaaaaaaaaaaaaaaaaaaaaaaaaaaaaaaaaaaaaaaaaaaaaaaaaaaaaaaaaaaaaaaaaa

SEQ ID NO: 20 - protein, 368 aa, mus musculus
MKSLWLCWALWVLPLAGPGAAMTEEQVLSSLLQQLQLSQAPTLDSADVEEMAIPTHVRSQYVALLQGSHADRSRGKRFSQNFREVAGRFLMSETSTHLLVFGMEQRLPPNSELVQAVLRLFQEPVPRTALRRFERLSPHSARARVTIEWLRVREDGSNRTALIDSRLVSIHESGWKAFDVTEAVNFWQQLSRPRQPLLLQVSVQREHLGPGTWSAHKLVRFAAQGTPDGKGQGEPQLELHTLDLKDYGAQGNCDPEVPVTEGTRCCRQEMYLDLQGMKWAENWILEPPGFLTYECVGSCLQLPESLTIGWPFLGPRQCVASEMTSLPMIVSVKEGGRTRPQVVSLPNMRVQTCSCASDGALIPRGIDL

SEQ ID NO: 21 - DNA, 9309 bp, homo sapiens
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgaaaaaaaaaaaaaaa

SEQ ID NO: 22 - protein, 2555 aa, homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 23 - DNA, 9193 bp, mus musculus
gtggtgtgcgtcaacgtccgatccccgccggccaccccaagaggccgccgcccgggctgcgggcagctggcgagcaggcatgccacggctcctgacgcccttgctctgcctaacgctgctgcccgcgctcgccgcaagaggcttgagatgctcccagccaagtgggacctgcctgaatggaggtaggtgcgaagtggccagcggcactgaagcctgtgtctgcagcggagcctttgtgggccaacgatgccaggactccaatccttgcctcagcacaccgtgtaagaatgctggaacgtgccacgttgtggaccatggtggcactgtggattatgcctgcagctgtcccctgggtttctctgggcccctctgcctgacacctctggacaacgcctgcctggccaacccctgccgcaatgggggcacctgtgacctgctcactctcacagagtacaagtgccgctgcccaccagggtggtcaggaaaatcatgtcagcaggctgacccctgtgcctccaacccctgtgccaatggtggccagtgcctgccctttgagtcttcatacatctgtcgctgcccgcctggcttccatggccccacctgcaggcaagatgttaatgagtgcagccagaaccctgggctgtgccgccatggaggcacctgccacaatgagatcggctcctatcgctgtgcctgccgtgccacccatactggtccccactgtgaactgccctatgtgccctgcagcccctcaccctgccagaatggaggcacctgccgtcctacaggggacaccacccacgagtgtgcctgcttgccaggttttgctggacagaactgtgaagaaaatgtggatgactgtccaggaaacaactgcaagaatgggggtgcctgtgtggacggcgtgaatacctacaattgccgctgcccaccggagtggacgggtcagtactgtacagaggatgtggacgaatgtcagctcatgcccaatgcctgccagaatggcggaacctgccacaacacacacggcggctacaactgtgtgtgtgtcaatgggtggactggcgaggactgcagtgagaacattgatgactgtgccagtgccgcctgtttccagggtgccacttgccacgaccgtgtggcttccttctactgcgaatgtccgcatgggcgcacaggtctgctgtgccacctcaacgatgcgtgcatcagcaacccctgcaacgagggctccaactgtgacaccaaccctgtcaacggcaaagccatctgcacctgcccctcggggtacacagggccagcctgcagccaggacgtggatgagtgtgctctgggtgccaacccttgtgagcacgcaggcaaatgcctcaacacactgggttcttttgagtgccagtgtctacagggctacacgggaccccgctgtgagattgatgttaatgagtgcatctccaacccatgtcagaatgatgccacttgcctggaccagattggggagttccaatgcatatgtatgccaggttatgaaggtgtatactgtgaaatcaacacggatgagtgcgccagcagcccctgtctgcacaatggccactgcatggacaagatcaatgagttccaatgtcagtgccccaaaggcttcaacgggcacctgtgccagtatgatgtggatgagtgtgccagcacaccatgcaagaacggtgccaagtgcctggatgggcccaacacctatacctgcgtgtgtacagaaggttacacagggacccactgcgaagtggacattgacgagtgtgaccctgacccctgccactatggttcctgtaaggatggtgtggccacctttacctgcctgtgccagccaggctacacaggccatcactgtgagaccaacatcaatgagtgccacagccaaccgtgccgccatgggggcacctgccaggaccgtgacaactcctacctctgcttatgcctcaagggaaccacagggcccaactgtgagatcaacctggatgactgcgccagcaacccctgtgactctggcacctgtctggacaagattgatggctacgaatgtgcctgtgaaccaggctacacaggaagcatgtgtaacgtcaacattgacgaatgtgcgggcagcccctgccacaacgggggcacttgtgaggatggcatcgcgggcttcacttgccgctgccccgagggctaccatgaccccacgtgcctgtccgaggtcaacgagtgcaacagtaacccctgcatccacggagcttgccgggatggcctcaatgggtacaagtgtgactgtgcccctgggtggagtggaacaaactgtgacatcaacaacaacgagtgtgagtccaacccttgtgtcaacggtggcacctgcaaggacatgaccagtggctacgtatgcacctgccgagaaggcttcagtggccctaattgccagaccaacatcaacgaatgtgcctccaacccctgcctgaaccaggggacctgcattgatgatgtcgctggatacaagtgcaactgtcctctgccatatacaggagccacgtgtgaggtggtgttggccccatgtgctaccagcccctgcaaaaacagcggggtatgcaaggagtctgaagactatgagagtttttcctgtgtctgtcccacaggctggcaaggtcaaacctgcgaggttgacatcaatgagtgtgtgaaaagcccatgtcgccatggggcctcctgccagaacaccaatggcagctaccgctgcctctgccaggccggctatacaggtcgcaactgtgagagtgacatcgatgactgccgccccaacccgtgtcacaatgggggttcctgcaccgatggcatcaacacagccttctgcgactgcctgcccggcttccagggtgccttctgtgaggaggacatcaatgaatgtgccagcaatccctgccaaaatggtgccaattgcactgactgtgtggacagctacacatgtacctgccccgtgggcttcaatggcatccactgcgagaacaacacacctgactgtactgagagctcctgcttcaatggtggtacctgtgtggatggtatcaactccttcacctgtctgtgtccacctggcttcacgggcagctactgtcagtatgatgtcaatgagtgtgattcacggccctgtctgcacggtggtacctgccaagacagctatggtacttataagtgtacctgcccacagggctacactggtctcaactgccagaaccttgtgcgctggtgcgactcggctccctgcaagaatggtggcaggtgctggcagaccaacacgcagtaccactgtgagtgccgcagcggctggactggcgtcaactgcgacgtgctcagtgtgtcctgtgaggtggctgcacagaagcgaggcattgacgtcactctcctgtgccagcatggagggctctgtgtggatgagggagataaacattactgccactgccaggcaggctacacgggcagctactgtgaggacgaggtggacgagtgctcacctaacccctgccagaatggagctacctgcactgactatctcggcggcttttcctgcaagtgtgtggctggctaccatgggtctaactgctcagaggagatcaacgagtgcctgtcccagccctgccagaatgggggtacctgcattgatctgaccaactcctacaagtgttcctgcccccgggggacacagggtgtacactgtgagatcaatgttgatgactgccatcccccccttgaccctgcctcccgaagccccaagtgcttcaacaatggcacctgtgtggaccaggtgggtggctatacctgcacctgcccaccaggcttcgtcggggagcggtgtgagggtgatgtcaatgaatgtctctccaacccctgtgacccacgtggcacccagaactgtgtgcagcgtgttaatgacttccactgcgagtgccgggctggccacactggacgccgctgtgagtcagtcatcaatggctgcaggggcaaaccttgcaagaatgggggtgtctgtgccgtggcctccaacaccgcccgtggattcatctgtaggtgccctgcgggcttcgagggtgccacatgtgagaatgatgcccgcacttgtggcagcttacgctgcctcaacggtggtacatgcatctcgggcccacgtagtcccacctgcctatgcctgggatccttcaccggccctgagtgccagttcccagccagcagcccctgtgtgggtagcaacccctgctacaatcagggcacctgtgagcccacatccgagaaccctttctaccgctgtctatgccctgccaaattcaacgggctactgtgccacatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggcagcatggccagctctggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacagacagtggacccagcagcacctggacgctgctgacctgcgcatgtctgccatggccccaacaccgcctcagggggaggtggatgctgactgcatggatgtcaatgttcgaggaccagatggcttcacacccctcatgattgcctcctgcagtggagggggccttgagacaggcaacagtgaagaagaagaagatgcacctgctgtcatctctgacttcatctaccagggcgccagcttgcacaaccagacagaccgcaccggggagaccgccttgcacttggctgcccgatactctcgttcagatgctgcaaagcgcttgctggaggccagtgcagatgccaacatccaggacaacatgggccgtactccgttacatgcagcagtttctgcagatgctcagggtgtcttccagatcctgctccggaacagggccacagatctggatgcccgaatgcatgatggcacaactccactgatcctggctgcgcgcctggccctggagggcatgctggaggacctcatcaactcacatgctgacgtcaatgccgtggatgacctaggcaagtcggctttgcattgggcggccgcggtgaacaatgtggatgctgctgttgtgctcctgaagaacggagccaacaaggacatgcagaacaacaaggaggagactcccctgttcctggccgcccgtgagggcagctatgagactgccaaagtgttgctggaccactttgccaaccgggacatcacggatcacatggaccgattgccgcgggacatcgcacaggagcgtatgcaccacgatatcgtgcggcttttggatgagtacaacctggtgcgcagcccacagctgcatggcactgccctgggtggcacacccactctgtctcccacactctgctcgcccaatggctacctgggcaatctcaagtctgccacacagggcaagaaggcccgcaagcccagcaccaaagggctggcttgtggtagcaaggaagctaaggacctcaaggcacggaggaagaagtcccaggatggcaagggctgcctgttggacagctcgagcatgctgtcgcctgtggactccctcgagtcaccccatggctacttgtcagatgtggcctcgccacccctcctcccctccccattccagcagtctccatccatgcctctcagccacctgcctggtatgcctgacactcacctgggcatcagccacttgaatgtggcagccaagcctgagatggcagcactggctggaggtagccggttggcctttgagccacccccgccacgcctctcccacctgcctgtagcctccagtgccagcacagtgctgagtaccaatggcacgggggctatgaatttcaccgtgggtgcaccggcaagcttgaatggccagtgtgagtggcttccccggctccagaatggcatggtgcccagccagtacaacccactacggccgggtgtgacgccgggcacactgagcacacaggcagctggcctccagcatagcatgatggggccactacacagcagcctctccaccaataccttgtccccgattatttaccagggcctgcccaacacacggctggcaacacagcctcacctggtgcagacccagcaggtgcagccacagaacttacagctccagcctcagaacctgcagccaccatcacagccacacctcagtgtgagctcggcagccaatgggcacctgggccggagcttcttgagtggggagcccagtcaggcagatgtacaaccgctgggccccagcagtctgcctgtgcacaccattctgccccaggaaagccaggccctgcccacatcactgccatcctccatggtcccacccatgaccactacccagttcctgacccctccttcccagcacagttactcctcctcccctgtggacaacacccccagccaccagctgcaggtgccagagcaccccttcctcaccccatcccctgagtcccctgaccagtggtccagctcctccccgcattccaacatctctgattggtccgagggcatctccagcccgcccaccaccatgccgtcccagatcacccacattccagaggcatttaaataaacagagatgtgggatgcaggaccccagcttccgttcccaagccctgttgggagtcctttccagtgcttcaggatgctggggcgaccaaaggagccttttaaaaaatgtttttatacaaaataagaggacaagaatttccatttttttttttagtatttatttatgtacttttattttccacagaaacactgcctttttatttatatgtattgttttctatggcactagggaaaaacatatctgttccaagaaaataaactagttctcagagccttgattttcctggtcagggtgaagttccctgtgtgtctgtaaaatatgaacaaggattcatgatttgtaaatgctgtttatttattgattgcttctttccaaaatcgaaaagaaagaaaaaagaacgtgacaggagaagggaagctggaaactgccatggccagaattgcccctcccccacactcactgcccctccccccagcgtcacctgggatttgcagatgtgtttagaaacacgcccagaccttgaaccttgggttcatggattagttttgtatctaaaacaggaaacaagtcagatgatgtggtttgtacactttctgtaaccaccagtgtggacttgaagaagtgtcctcagcatgtgcagagtctactacccagtaccagtcgtgagtctgcaggctccagtgttctgtagtagtgtttatgggccttgggagtacttctcccctgccctgccccactgtccccttcctgacaacttgagccagtaagccatgcagggtgtggtgcctcctagagaaaacactgcctggactgttctgtgcatccctccaaacagcatcatccaaatccaactgaggacagacggactgtcccggcctgggcctgggctcctaacacctgactgccaaagggctccaatgtgcattgtggactcgccagagtagcctgcattgagactccaagaaaacagaagctatgtggcctctgatccccaaactggcctgggtggggacatgccttgagtgtgctggaatgtgggtggagcctgcttctgggccacccctcctggttcagggctgtgctcacagcagattcttgcagtatcaagtatacgcctgtggcagaataagtatctgtaaatacatgtttaaagaggattttgtttaaaaaatctaaaggaacaagtgtgtcgtgtgtcaagctgatgaggactgtcagactgtggcttagctcagtgtgacccagaccttgtgacctgtagctgccgaaccagtagctcctaagagcacaacccaggatggcccatctgctgcccaccaagtccctttccagccactgtgtgctgggggctttcgggggcagttgcccacctcctcagggcagctctttctggccttttggggggcagtgtctgtgccatgcctaatagatatgaccagacgcatcctaagatgttgattcttactgtgttgtataaaataaagtgtagtttacaaaaaaaagaaaaaaaaaaaaaaa

SEQ ID NO: 24 - protein, 2531 aa, mus musculus
MPRLLTPLLCLTLLPALAARGLRCSQPSGTCLNGGRCEVASGTEACVCSGAFVGQRCQDSNPCLSTPCKNAGTCHVVDHGGTVDYACSCPLGFSGPLCLTPLDNACLANPCRNGGTCDLLTLTEYKCRCPPGWSGKSCQQADPCASNPCANGGQCLPFESSYICRCPPGFHGPTCRQDVNECSQNPGLCRHGGTCHNEIGSYRCACRATHTGPHCELPYVPCSPSPCQNGGTCRPTGDTTHECACLPGFAGQNCEENVDDCPGNNCKNGGACVDGVNTYNCRCPPEWTGQYCTEDVDECQLMPNACQNGGTCHNTHGGYNCVCVNGWTGEDCSENIDDCASAACFQGATCHDRVASFYCECPHGRTGLLCHLNDACISNPCNEGSNCDTNPVNGKAICTCPSGYTGPACSQDVDECALGANPCEHAGKCLNTLGSFECQCLQGYTGPRCEIDVNECISNPCQNDATCLDQIGEFQCICMPGYEGVYCEINTDECASSPCLHNGHCMDKINEFQCQCPKGFNGHLCQYDVDECASTPCKNGAKCLDGPNTYTCVCTEGYTGTHCEVDIDECDPDPCHYGSCKDGVATFTCLCQPGYTGHHCETNINECHSQPCRHGGTCQDRDNSYLCLCLKGTTGPNCEINLDDCASNPCDSGTCLDKIDGYECACEPGYTGSMCNVNIDECAGSPCHNGGTCEDGIAGFTCRCPEGYHDPTCLSEVNECNSNPCIHGACRDGLNGYKCDCAPGWSGTNCDINNNECESNPCVNGGTCKDMTSGYVCTCREGFSGPNCQTNINECASNPCLNQGTCIDDVAGYKCNCPLPYTGATCEVVLAPCATSPCKNSGVCKESEDYESFSCVCPTGWQGQTCEVDINECVKSPCRHGASCQNTNGSYRCLCQAGYTGRNCESDIDDCRPNPCHNGGSCTDGINTAFCDCLPGFQGAFCEEDINECASNPCQNGANCTDCVDSYTCTCPVGFNGIHCENNTPDCTESSCFNGGTCVDGINSFTCLCPPGFTGSYCQYDVNECDSRPCLHGGTCQDSYGTYKCTCPQGYTGLNCQNLVRWCDSAPCKNGGRCWQTNTQYHCECRSGWTGVNCDVLSVSCEVAAQKRGIDVTLLCQHGGLCVDEGDKHYCHCQAGYTGSYCEDEVDECSPNPCQNGATCTDYLGGFSCKCVAGYHGSNCSEEINECLSQPCQNGGTCIDLTNSYKCSCPRGTQGVHCEINVDDCHPPLDPASRSPKCFNNGTCVDQVGGYTCTCPPGFVGERCEGDVNECLSNPCDPRGTQNCVQRVNDFHCECRAGHTGRRCESVINGCRGKPCKNGGVCAVASNTARGFICRCPAGFEGATCENDARTCGSLRCLNGGTCISGPRSPTCLCLGSFTGPECQFPASSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLALEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACGSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLQLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSPHSNISDWSEGISSPPTTMPSQITHIPEAFK

SEQ ID NO: 25 - DNA, 1744 bp, homo sapiens
atgcacgcccactgcctgcccttccttctgcacgcctggtgggccctactccaggcgggtgctgcgacggtggccactgcgctcctgcgtacgcgggggcagccctcgtcgccatcccctctggcgtacatgctgagcctctaccgcgacccgctgccgagggcagacatcatccgcagcctacaggcagaagatgtggcagtggatgggcagaactggacgtttgcttttgacttctccttcctgagccaacaagaggatctggcatgggctgagctccggctgcagctgtccagccctgtggacctccccactgagggctcacttgccattgagattttccaccagccaaagcccgacacagagcaggcttcagacagctgcttagagcggtttcagatggacctattcactgtcactttgtcccaggtcaccttttccttgggcagcatggttttggaggtgaccaggcctctctccaagtggctgaagcgccctggggccctggagaagcagatgtccagggtagctggagagtgctggccgcggccccccacaccgcctgccaccaatgtgctccttatgctctactccaacctctcgcaggagcagaggcagctgggtgggtccaccttgctgtgggaagccgagagctcctggcgggcccaggagggacagctgtcctgggagtggggcaagaggcaccgtcgacatcacttgccagacagaagtcaactgtgtcggaaggtcaagttccaggtggacttcaacctgatcggatggggctcctggatcatctaccccaagcagtacaacgcctatcgctgtgagggcgagtgtcctaatcctgttggggaggagtttcatccgaccaaccatgcatacatccagagtctgctgaaacgttaccagccccaccgagtcccttccacttgttgtgccccagtgaagaccaagccgctgagcatgctgtatgtggataatggcagagtgctcctagatcaccataaagacatgatcgtggaagaatgtgggtgcctctgatgacatcctggagggagactggatttgcctgcactctggaaggctgggaaactcctggaagacatgataaccatctaatccagtaaggagaaacagagaggggcaaagttgctctgcccaccagaactgaagaggaggggctgcccactctgtaaatgaagggctcagtggagtctggccaagcacagaggctgctgtcaggaagagggaggaagaagcctgtgcagggggctggctggatgttctctttactgaaaagacagtggcaaggaaaagcacaagtgcatgagttctttactggattttttaaaaacctgtgaaccccccgaaactgtatgtgaaagttgagacatatgtgcatgtattttggaggtgggatgaagtcacctatagctttcatgtattctccaaagtagtctgtgtgtgacctgtccccctccccaaagattaaggatcactgtatagattaaaaagagtccgtcaatctcattgcctcaggctgggttgggggagccccacagctttctggctggccagtggcaatctactggccttgtccagaggctcactggagtggttctctgctaatgagctgtacaacaataaagccattgtctagttaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - protein, 347 aa, homo sapiens
MHAHCLPFLLHAWWALLQAGAATVATALLRTRGQPSSPSPLAYMLSLYRDPLPRADIIRSLQAEDVAVDGQNWTFAFDFSFLSQQEDLAWAELRLQLSSPVDLPTEGSLAIEIFHQPKPDTEQASDSCLERFQMDLFTVTLSQVTFSLGSMVLEVTRPLSKWLKRPGALEKQMSRVAGECWPRPPTPPATNVLLMLYSNLSQEQRQLGGSTLLWEAESSWRAQEGQLSWEWGKRHRRHHLPDRSQLCRKVKFQVDFNLIGWGSWIIYPKQYNAYRCEGECPNPVGEEFHPTNHAYIQSLLKRYQPHRVPSTCCAPVKTKPLSMLYVDNGRVLLDHHKDMIVEECGCL

SEQ ID NO: 27 - DNA, 1065 bp, mus musculus
atgagtgcccacagcctccgcatccttcttcttcaagcctgttgggctctactccacccgcgcgccccgaccgcggccgctttgcctctgtggacacgggggcagccctcgtcaccgtcccctctggcgtacatgttgagcctctaccgagacccgctgcctcgggcggacatcatccgcagcctccaggcgcaagatgtggacgtgaccggacagaactggactttcacgtttgacttctcctttttgagccaagaagaggatctggtatgggcggacgtccggttgcagctgccgggccccatggacatacccactgagggcccactcaccattgacattttccaccaggccaagggggatccagagcgggaccccgctgactgcctggagcgcatttggatggagacgttcaccgtcattccttctcaggtcacgtttgcctcaggcagcacagtcctggaggtgaccaagccactctccaagtggctaaaggaccccagggcactggaaaagcaggtgtccagtcgagcagaaaagtgttggcatcagccctacaccccacctgtacctgtcgccagcaccaatgtgctcatgctctactccaaccggcctcaggagcagaggcagctagggggcgccactttgctttgggaagctgagagctcctggcgggcccaggagggacagctgtctgtagagaggggcggatggggcagaaggcaacgccgacatcatttgccagacagaagccaactgtgtaggagggtcaagttccaggtggacttcaacctgattggctggggctcctggatcatctaccccaagcagtacaatgcctatcgctgtgagggcgagtgtcctaaccctgtgggggaggagtttcatcctaccaaccatgcctacatccagagcctgctgaaacgataccaaccccaccgggttccttccacgtgctgtgcccccgtgaagaccaagccactgagcatgctttatgtggacaatggcagggtcctcctggaacaccacaaggacatgattgtggaggagtgtgggtgcctctga

SEQ ID NO: 28 - protein, 354 aa, mus musculus
MSAHSLRILLLQACWALLHPRAPTAAALPLWTRGQPSSPSPLAYMLSLYRDPLPRADIIRSLQAQDVDVTGQNWTFTFDFSFLSQEEDLVWADVRLQLPGPMDIPTEGPLTIDIFHQAKGDPERDPADCLERIWMETFTVIPSQVTFASGSTVLEVTKPLSKWLKDPRALEKQVSSRAEKCWHQPYTPPVPVASTNVLMLYSNRPQEQRQLGGATLLWEAESSWRAQEGQLSVERGGWGRRQRRHHLPDRSQLCRRVKFQVDFNLIGWGSWIIYPKQYNAYRCEGECPNPVGEEFHPTNHAYIQSLLKRYQPHRVPSTCCAPVKTKPLSMLYVDNGRVLLEHHKDMIVEECGCL

SEQ ID NO: 29 - DNA, 18 bp, artificial
  Oligonucleotide primer
ggaaatcgtgcgtgacat

SEQ ID NO: 30 - DNA, 23 bp, artificial
  Oligonucleotide primer
tcatgatggagttgaatgtagtt

SEQ ID NO: 31 - DNA, 19 bp, artificial
  Oligonucleotide primer
cgcggggatccgaggactg

SEQ ID NO: 32 - DNA, 20 bp, artificial
  Oligonucleotide primer
tacacaggatgggtcgtaca

SEQ ID NO: 33 - DNA, 20 bp, artificial
  Oligonucleotide primer
cgtgaggtcccagtatgtgg

SEQ ID NO: 34 - DNA, 20 bp, artificial
  Oligonucleotide primer
gtagtccttgaggtccagcg

SEQ ID NO: 35 - DNA, 20 bp, artificial
  Oligonucleotide primer
gtgaccggacagaactggac

SEQ ID NO: 36 - DNA, 20 bp, artificial
  Oligonucleotide primer
ctgtctggcaaatgatgtcg

SEQ ID NO: 37 - DNA, 19 bp, artificial
  Oligonucleotide primer
aggttcagccagagcttcc

SEQ ID NO: 38 - DNA, 18 bp, artificial
  Oligonucleotide primer
caccagcaggtgtgtgct

SEQ ID NO: 39 - DNA, 20 bp, artificial
  Oligonucleotide primer
cctggacctcagggactatg

SEQ ID NO: 40 - DNA, 20 bp, artificial
  Oligonucleotide primer
atcccctgcaggtcaatgta

SEQ ID NO: 41 - DNA, 19 bp, artificial
  Oligonucleotide primer
agacatcatccgcagccta

SEQ ID NO: 42 - DNA, 21 bp, artificial
  Oligonucleotide primer
caaaagcaaacgtccagttct

SEQ ID NO: 43 - DNA, 18 bp, artificial
  Oligonucleotide primer
ccttacggaagcccgagt

SEQ ID NO: 44 - DNA, 19 bp, artificial
  Oligonucleotide primer
ccgaagccattcttgcata

SEQ ID NO: 45 - DNA, 18 bp, artificial
  Oligonucleotide primer
cctgctgccctacacagg

SEQ ID NO: 46 - DNA, 22 bp, artificial
  Oligonucleotide primer
agctctcatagtcctcggattg

SEQ ID NO: 47 - DNA, 21 bp, artificial
  Oligonucleotide primer
actcagtatgtggccctgcta

SEQ ID NO: 48 - DNA, 18 bp, artificial
  Oligonucleotide primer
aacctgcctgccacctct

SEQ ID NO: 49 - DNA, 19 bp, artificial
  Oligonucleotide primer
cacaagttggtccgtttcg

SEQ ID NO: 50 - DNA, 19 bp, artificial
  Oligonucleotide primer
ggtacctcggggtcacaat

SEQ ID NO: 51 - DNA, 19 bp, artificial
  Oligonucleotide primer
ccaaccatgcctacatcca

SEQ ID NO: 52 - DNA, 19 bp, artificial
  Oligonucleotide primer
cacagcacgtggaaggaac

SEQ ID NO: 53 - DNA, 22 bp, artificial
  Oligonucleotide primer
gactcatttcactagccagcag

SEQ ID NO: 54 - DNA, 18 bp, artificial
  Oligonucleotide primer
cggcgattcttgaaccaa

SEQ ID NO: 55 - DNA, 20 bp, artificial
  Oligonucleotide primer
ccaacaaggacatgcagaac

SEQ ID NO: 56 - DNA, 20 bp, artificial
  Oligonucleotide primer
cagtctcatagctgccctca

SEQ ID NO: 57 - DNA, 20 bp, artificial
  Oligonucleotide primer
ctacaggtgtcggtgcagag

SEQ ID NO: 58 - DNA, 22 bp, artificial
  Oligonucleotide primer
aagtccctcgatggctacacta

SEQ ID NO: 59 - DNA, 20 bp, artificial
  Oligonucleotide primer
cgtccatcacccatcctaag

SEQ ID NO: 60 - DNA, 20 bp, artificial
  Oligonucleotide primer
cgtccatcacccatcctaag

SEQ ID NO: 61 - DNA, 24 bp, artificial
  Oligonucleotide primer
atcatctaccccaagcagtacaac

SEQ ID NO: 62 - DNA, 24 bp, artificial
  Oligonucleotide primer
actgagcccttcatttacagagtg

SEQ ID NO: 63 - DNA, 22 bp, artificial
  Oligonucleotide primer
gaggacccgtctaagaagaagc

SEQ ID NO: 64 - DNA, 24 bp, artificial
  Oligonucleotide primer
tcaagttattcaggctgttgagac

SEQ ID NO: 65 - DNA, 24 bp, artificial
  Oligonucleotide primer
cagaactgtgaggaaaatatcgac

SEQ ID NO: 66 - DNA, 20 bp, artificial
  Oligonucleotide primer
agttggagccctcgttacag

SEQ ID NO: 67 - DNA, 18 bp, artificial
  Oligonucleotide primer
tcagcctgcccaacatga

SEQ ID NO: 68 - DNA, 21 bp, artificial
  Oligonucleotide primer
ttcacatctagcaaagccagt

SEQ ID NO: 69 - DNA, 20 bp, artificial
  Oligonucleotide primer
cgtgaggtcccagtatgtgg

SEQ ID NO: 70 - DNA, 20 bp, artificial
  Oligonucleotide primer
gtagtccttgaggtccagcg

SEQ ID NO: 71 - DNA, 20 bp, artificial
  Oligonucleotide primer
gtgaccggacagaactggac

SEQ ID NO: 72 - DNA, 20 bp, artificial
  Oligonucleotide primer
ctgtctggcaaatgatgtcg